(19) United States
(12) Patent Application Publication
(10) Pub. No.: US 2006/0099600 A1
(43) Pub. Date: May 11, 2006

(54) Novel acetylcholine transporter

(75) Inventor: Steven McIntire, Tiburon, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 10/868,498

(22) Filed: Jun. 14, 2004

Related U.S. Application Data
(60) Provisional application No. 60/480,508, filed on Jun. 20, 2003.

(51) Int. Cl. C12Q 1/68; G01N 33/53; C07H 21/04; C12P 21/06; C07K 14/705; C07K 16/28

(52) U.S. Cl. 435/6; 435/7.1; 435/69.1; 435/320.1; 435/325; 530/350; 536/23.5; 530/388.22

(57) ABSTRACT

This invention provides novel acetylcholine transporters. The transporters are effective and useful targets to screen for modulators of cholinergic synaptic activity. Also provided are methods of modulating the activity of cholinergic synapses using modulator of acetylcholine transporter expression and/or activity.

### CROSS-REFERENCE TO RELATED APPLICATIONS

This application claims priority to and benefit of U.S. Ser. No. 60/480,508, filed Jun. 20, 2003 which is incorporated herein by reference in its entirety for all purposes.

### STATEMENT AS TO RIGHTS TO INVENTIONS MADE UNDER FEDERALLY SPONSORED RESEARCH AND DEVELOPMENT

[Not Applicable]

### FIELD OF THE INVENTION

This invention pertains to the field of neurobiology. In particular this invention pertains to the identification of novel acetylcholine transporters.

### BACKGROUND OF THE INVENTION

The cholinergic transmissions or neuromodulations in the central nervous system are involved in a number of fundamental brain processes such as learning and memory (Aigner & Mishkin(1986) Behav. & Neural. Biol. 45: 81-87; Fibinger (1991) TINS, 14:220-223), arousal, and sleep-wake cycles (Karczmar (1976) Pp. 395-449 In: Biology of Cholinergic Function, (eds A. M. Goldberg & I. Hanin) Raven Press, N.Y.). In this system, the formation of the neurotransmitter acetylcholine is catalyzed by the enzyme choline acetyltransferase (ChAT, E.C. 2.3.1.6), which transfers an acetyl group from acetylcoenzyme A to choline, in the presynaptic nerve terminals of cholinergic neurons. Acetylcholine is packaged into the synaptic vesicles by a vesicular acetylcholine transporter (VAChT) and is then ready to be released in a calcium dependent manner. Acetylcholine binds specifically to either the nicotinic or muscarinic receptors (AChR) to transmit information to the postsynaptic neurons. The action of acetylcholine is terminated through hydrolysis to acetate and choline by the enzyme acetylcholinesterase. Most of the choline is then transported back to the presynaptic terminal to be recycled as one of the precursors for the biosynthesis of acetylcholine. This step, which is mediated by the action of the high affinity choline transporter (HACT), is believed to be the rate limiting step of the biosynthesis of the neurotransmitter acetylcholine, which plays a pivotal role in processes such as learning, memory, and sleep (Srinivasan et al. (1976) Biochem. Pharmacol. 25(24): 2739-2745.).

Altered functioning of the cholinergic system has been observed during normal aging processes (Cohen et al. (1995) JAMA, 274: 902-907; Smith et al. (1995) Neurobiol Aging, 16: 161-73 (1995)), while its dysfunction underlies nicotine addiction and a number of neurological and psychiatric disorders most notably Alzheimer's disease (AD), Myasthenia Gravis, Amyotrophic Lateral Sclerosis (ALS), and epilepsies.

### SUMMARY OF THE INVENTION

This invention pertains to the discovery of novel acetylcholine transporters. The transporters are effective and useful targets to screen for modulators of cholinergic synaptic activity. Such modulators are effective in a number of neuropathologies and in certain other contexts, e.g. as described herein.

Definitions

The terms “polypeptide”, “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers. The term also includes variants on the traditional peptide linkage joining the amino acids making up the polypeptide.

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al. (1993) Tetrahedron 49(10):1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 1419), phosphorothioate (Mag et al. (1991) Nucleic Acids Res. 19:1437; and U.S. Pat. No. 5,644,048), phosphorodithioate (Briu et al. (1989) J. Am. Chem. Soc. 111:2321, O-methylphophoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Sanghui and Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Sanghui and Cook. Nucleic acids containing one or more carbocyclic sugars are also included within the definition of nucleic acids (see Jenkins et al. (1995), Chem. Soc. Rev. pp 169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

An acetylcholine transporter nucleic acid refers to a nucleic acid that encodes an acetylcholine transporter. Such acetylcholine transporter nucleic acids include, but are not limited to the C. elegans acetylcholine transporter and/or the homologues or orthologues thereof identified herein, or to a nucleic acid derived therefrom. Thus acetylcholine transporter nucleic acids include, but are not limited, to an acetylcholine transporter gene, an acetylcholine transporter cDNA, a n acetylcholine transporter RNA, a n acetylcholine transporter cRNA, an amplification produce produced from an acetylcholine transporter nucleic acid template, and the like.

The phrase “detecting expression or activity of nn acetylcholine transporter” refers to detecting expression of an acetylcholine transporter nucleic acid, detecting expression of a n acetylcholine transporter polypeptide, or detecting activity of an acetylcholine transporter polypeptide.

The term “inhibit expression” when used with reference to inhibition of an acetylcholine transporter refers to a reduction or blocking of VGLUT transcription, and/or translation, and/or formation or availability or activity of a n acetylcholine transporter protein.

The term “detecting an acetylcholine transporter mRNA or cDNA” refers to detecting and/or quantifying an acetylcholine transporter nucleic acid or a nucleic acid derived therefrom the quantification of which provides an indication of the expression level of the acetylcholine transporter nucleic acid. The term thus includes, but is not limited to detection of acetylcholine transporter mRNA, cDNA, acetylcholine transporter amplification products, and fragments of any of these.

The terms “binding partner”, or “capture agent”, or a member of a “binding pair” refers to molecules that specifically bind other molecules to form a binding complex such as antibody-antigen, lectin-carbohydrate, nucleic acid-nucleic acid, biotin-avidin, etc.

The term “specifically binds”, as used herein, when referring to a biomolecule (e.g., protein, nucleic acid, antibody, etc.), refers to a binding reaction which is determinative of the presence biomolecule in heterogeneous population of molecules (e.g., proteins and other biologics). Thus, under designated conditions (e.g. immunoassay conditions in the case of an antibody or stringent hybridization conditions in the case of a nucleic acid), the specified ligand or antibody binds to its particular “target” molecule and does not bind in a significant amount to other molecules present in the sample.

The phrase “transport of acetylcholine into a cell” refers to the uptake of acetylcholine into, e.g., a synaptic vesicle (e.g. of a nerve cell), or the uptake of acetylcholine into other kinds of cells, as well. Thus, for example, transport of acetylcholine into a cell can refer to the transport of acetylcholine into an oocyte (e.g., an oocytes expressing a heterologous acetylcholine transporter) in which case, uptake is across the plasma membrane. In certain preferred embodiments, uptake is uptake by a mammalian cell.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all to, other sequences. Stringent hybridization and stringent hybridization wash conditions in the context of nucleic acid hybridization are sequence dependent, and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part I, chapt 2, Overview of principles of hybridization and the strategy of nucleic acid probe assays, Elsevier, N.Y. (Tijssen). Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or on a filter in a Southern or northern blot is 42° C. using standard hybridization solutions (see, e.g., Sambrook (1989) Molecular Cloning: A Laboratory Manual (2nd ed.) Vol. 1-3, Cold Spring Harbor Laboratory, Cold Spring Harbor Press, NY, and detailed discussion, below), with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, e.g., Sambrook supra.) for a description of SSC buffer). Often, a high stringency wash is preceded by a low stringency wash to remove background probe signal. An example medium stringency wash for a duplex of, e.g., more than 100 nucleotides, is 1×SSC at 45° C. for 15 minutes. An example of a low stringency wash for a duplex of, e.g., more than 100 nucleotides, is 4× to 6×SSC at 40° C. for 15 minutes.

The term “test agent” refers to an agent that is to be screened in one or more of the assays described herein. The agent can be virtually any chemical compound. It can exist as a single isolated compound or can be a member of a chemical (e.g. combinatorial) library. A test agents can be a pharmacological agent already known in the art or can be a compound previously unknown to have any pharmacological activity. The agents can be naturally occurring or designed in the laboratory. It cam be isolated from microorganisms, animals, or plants, can be produced recombinantly, or synthesized by chemical methods known in the art. If desired, test agents can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, synthetic library methods using affinity chromatography selection, and the like. The biological library approach is often limited to polypeptide libraries, while the other four approaches are applicable to polypeptide, non-peptide oligomer, or small molecule libraries of compounds (see, e.g., Lam (1997) Anticancer Drug Des. 12: 145). In a particularly preferred embodiment, the test agent will be a small organic molecule.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

The term “database” refers to a means for recording and retrieving information. In preferred embodiments the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

The term “heterologous” as it relates to nucleic acid sequences such as coding sequences and control sequences, denotes sequences that are not normally associated with a region of a recombinant construct, and/or are not normally associated with a particular cell. Thus, a “heterologous” region of a nucleic acid construct is an identifiable segment of nucleic acid within or attached to another nucleic acid molecule that is not found in association with the other molecule in nature. For example, a heterologous region of a construct could include a coding sequence flanked by sequences not found in association with the coding sequence in nature. Another example of a heterologous coding sequence is a construct where the coding sequence itself is not found in nature (e.g., synthetic sequences having codons different from the native gene). Similarly, a host cell transformed with a construct which is not normally present in the host cell would be considered heterologous for purposes of this invention.

The term “recombinant” or “recombinantly expressed” when used with reference to a cell indicates that the cell replicates or expresses a nucleic acid, or expresses a peptide or protein encoded by a nucleic acid whose origin is exogenous to the cell. Recombinant cells can express genes that are not found within the native (non-recombinant) form of the cell. Recombinant cells can also express genes found in the native form of the cell wherein the genes are re-introduced into the cell by artificial means, for example under the control of a heterologous promoter.

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection. With respect to the peptides of this invention sequence identity is determined over the full length of the peptide.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman (1988) Proc. Natl. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle (1987) J. Mol. Evol. 35:351-360. The method used is similar to the method described by Higgins & Sharp (1989) CABIOS 5: 151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described in Altschul et al. (1990) J. Mol. Biol. 215: 403-410. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul (1993) Proc. Natl. Acad. Sci. USA,90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

The term “operably linked” as used herein refers to linkage of a promoter to a nucleic acid sequence such that the promoter mediates/controls transcription of the nucleic acid sequence.

The term “induce” expression refers to an increase in the transcription and/or translation of a gene or cDNA.

### BRIEF DESCRIPTION OF THE DRAWINGS DETAILED DESCRIPTION

Synpatic transmission at cholinergic synapses in the brain and peripheral nervous system involves regulated release of acetylcholine and metabolism of acetylcholine by acetylcholinesterases. We have identified an additional component of cholinergic synapses—a novel plasma membrane acetylcholine transporter in C. elegans (see, e.g., SEQ ID NO:1 and SEQ ID NO:2). The transporter is localized to cholinergic synapses and is required during periods of elevated synaptic activity to remove acetylcholine from the synaptic cleft. A plasma membrane transporter of acetylcholine has not been previously described in any system. The transporter that we have identified is similar to other Na⁺ and Cl⁻ dependent neurotransmitter transporters such as the dopamine transporter, DAT, and the GABA transporter, GAT. Many of these plasma membrane neurotransmitter transporters have proven to be extremely important therapeutic targets. For instance the antidepressants modulate dopaminergic function through an effect on DAT.

Cholinergic synapses are essential for the normal function of the mammalian brain and peripheral nervous system are also thought to be involved in multiple pathological conditions. Cholinergic neurons are critical in learning and memory and defects in cholinergic function have been correlated with severity of dementia. There is evidence of abnormal cholinergic function in Alzheimer's disease, Down's syndrome, Parkinson's disease, and schizophrenia. Cholinergic function is also disrupted in peripheral nerve and muscular disease, such as the muscular dystrophies and myasthenia gravis. Finally, cholinergic function has been implicated in drug addiction, including addiction to nicotine, ethanol, neurostimulants (such as cocaine and amphetamine) and opiates. The identification of a plasma membrane transporter of acetylcholine now provides a means of pharmacologically modulating cholinergic function in multiple disease states, including but not limited to all of the above conditions.

In addition to the acetylcholine transporter identified in C. elegans, we have identified multiple possible vertebrate orthologues of this transporter. The genbank or Celera numbers of all of these transporters are provided in Table 1.

TABLE 1
Table 1. Acetylcholine transporter orthologues.

| | | |
|---|---|---|
| gi\|4759136\|ref\|NP_004202.1\| | solute carrier family 6 (neurotransmitter | |
| | transporter, glycine), member 5; SLC6A5 solute carrier family 6 | |
| | (neurotransmitter transporter, glycine), member 5 [Homo sapiens] | |
| gi\|17380317\|sp\|Q9Y345\|S6A5_HUMAN | Sodium- and chloride-dependent glycine | |
| | transporter 2 (GlyT2) (GlyT-2) | |
| gi\|4003525\|gb\|AAC95145.1\| | glycine transporter GLYT2 [Homo sapiens] | |
| gi\|13122804\|gb\|AAK12641.1\|AF117999_1 | sodium- and chloride-dependent | |
| | glycine transporter type II [Homo sapiens] Length = 797 | |
| gi\|4689410\|gb\|AAD27892.1\|AF142501_1 | glycine transporter type-2 [Homo | |
| | sapiens] Length = 797 | |
| gi\|13549154\|gb\|AAK29670.1\|AF352733_1 | glycine type 2 transporter variant | |
| | SC6 [Homo sapiens] Length = 797 | |
| gi\|1352532\|sp\|P48067\|S6A9_HUMAN | Sodium- and chloride-dependent glycine | |
| | transporter 1 (GlyT1) (GlyT-1) | |
| gi\|2119585\|pir\|\|I57956 | glycine transporter type 1b - human | |
| gi\|546769\|gb\|AAB30784.1\| | glycine transporter type 1b; GlyT-1b [Homo | |
| | sapiens]Length = 692 | |
| gi\|6005715\|ref\|NP_009162.1\| | solute carrier family 6 (neurotransmitter | |
| | transporter), member 14; amino acid transporter B0+ [Homo sapiens] | |
| gi\|5732680\|gb\|AAD49223.1\|AF151978_1 | amino acid transporter B0+ [Homo | |
| | sapiens] Length = 642 | |
| gi\|7657589\|ref\|NP_055043.1\| | solute carrier family 6, member 7; brain- | |
| | specific L-proline transporter [Homo sapiens] | |
| gi\|3024229\|sp\|Q99884\|S6A7_HUMAN | Sodium-dependent proline transporter | |
| gi\|8176779\|gb\|AAB47007.2\| | brain-specific L-proline transporter [Homo | |
| | sapiens] Length = 636 | |
| gi\|27715467\|ref\|XP_233305.1\| | similar to solute carrier family 6 | |
| | (neurotransmitter transporter), member 14; amino acid transporter B0+ | |
| | [Homo sapiens] [Rattus norvegicus] Length = 558 | |
| gi\|14161715\|emb\|CAC39181.1\| | alternative [Homo sapiens] Length = 628 | |
| gi\|4557046\|ref\|NP_001034.41\| | solute carrier family 6 (neurotransmitter | |
| | transporter, noradrenalin), member 2; noradrenaline transporter; solute | |
| | carrier family 6 (neurotransmitter transporter, norepinephrine), member | |
| | 5; norepinephrine transporter [Homo sapiens] | |
| gi\|128616\|sp\|P23975\|S6A2_HUMAN | Sodium-dependent noradrenaline | |
| | transporter (Norepinephrine transporter) (NET) | |
| gi\|107214\|pir\|\|S14278 | noradrenaline transport protein - human | |
| gi\|189258\|gb\|AAA59943.1\| | noradrenaline transporter | |
| gi\|1143479\|emb\|CAA62566.1\| | norepinephrine transporter [Homo sapiens] | |
| gi\|227608\|prf\|\|1707305A | noradrenaline transporter Length = 617 | |
| gi\|7108463\|gb\|AAC50179.2\| | dopamine transporter [Homo sapiens] Length = 620 | |
| gi\|21707908\|gb\|AAH33904.1\| | solute carrier family 6 (neurotransmitter | |
| | transporter, GABA), member 1 [Homo sapiens] Length = 599 | |
| gi\|7657587\|ref\|NP_055044.1\| | solute carrier family 6 (neurotransmitter | |
| | transporter, GABA), member 11 [Homo sapiens] | |
| gi\|1352531\|sp\|P48066\|S6AB_HUMAN | Sodium- and chloride-dependent GABA | |
| | transporter 3 | |
| gi\|913242\|gb\|AAB33570.1\| | gamma-aminobutyric acid transporter type 3; | |
| | GABA transporter type 3; GAT-3 [Homo sapiens] Length = 632 | |
| gi\|19923157\|ref\|NP_003035.2\| | solute carrier family 6 (neurotransmitter | |
| | transporter, betaine/GABA), member 12; gamma-aminobutyric acid | |
| | transporter [Homo sapiens] | |
| gi\|2134824\|pir\|\|S68236 | betaine/GABA transport protein BGT-1 - human | |
| gi\|881475\|gb\|AAA87029.1\| | pephBGT-1 betaine-GABA transporter Length = 614 | |
| gi\|1352525\|sp\|P48065\|S6AC_HUMAN | Sodium- and chloride-dependent betaine | |
| | transporter (Na+/Cl−betaine/GABA transporter) (BGT-1) | |
| gi\|808696\|gb\|AAA66574.1\| | betaine/GABA transporter Length = 614 | |
| >gi\|5032097\|ref\|NP_005620.1\| | solute carrier family 6 (neurotransmitter | |
| | transporter, creatine), member 8 [Homo sapiens] | |
| gi\|1352529\|sp\|P48029\|S6A8_HUMAN | Sodium- and chloride-dependent creatine | |
| | transporter 1 (CT1) | |
| gi\|7441658\|pir\|\|G02095 | creatine transporter - human | |
| gi\|1020319\|gb\|AAA79507.1\| | creatine transporter | |
| gi\|1628387\|emb\|CAA91442.1\| | creatine transporter [Homo sapiens] | |
| gi\|15214460\|gb\|AAH12355.1\|AAH12355 | Similar to solute carrier family 6 | |
| | (neurotransmitter transporter, creatine), member 8 [Homo sapiens] Length = 635 | |
| gi\|4507039\|ref\|NP_003033.1\| | solute carrier family 6 (neurotransmitter | |
| | transporter, GABA), member 1 [Homo sapiens] | |
| gi\|266666\|sp\|P30531\|S6A1_HUMAN | Sodium- and chloride-dependent GABA | |
| | transporter 1 | |
| gi\|106051\|pir\|\|S11073 | gamma-aminobutyric acid transport protein - human | |
| gi\|31658\|emb\|CAA38484.1\| | GABA transporter [Homo sapiens] Length = 599 | |
| gi\|4507041\|ref\|NP_001035.1\| | solute carrier family 6 (neurotransmitter | |
| | transporter, dopamine), member 3; dopamine transporter [Homo sapiens] | |
| gi\|266667\|sp\|Q01959\|S6A3_HUMAN | Sodium-dependent dopamine transporter (DA | |
| | transporter) (DAT) | |
| gi\|477412\|pir\|\|A48980 | dopamine transporter - human | |
| gi\|181656\|gb\|AAC41720.1\| | dopamine transporter | |
| gi\|258935\|gb\|AAA11754.1\| | dopamine transporter [Homo sapiens] | |
| gi\|401765\|gb\|AAA19560.1\| | dopamine transporter | |
| gi\|2447032\|dbj\|BAA22511.1\| | dopamine transporter [Homo sapiens] | |
| gi\|11275971\|gb\|AAG33844.1\| | dopamine transporter [Homo sapiens] | |
| | Length = 620 | |
| gi\|2119587\|pir\|\|I57937 | dopamine transporter - human | |
| gi\|256313\|gb\|AAB23443.1\| | dopamine transporter; DAT [Homo sapiens] Length = 620 | |
| gi\|21361581\|ref\|NP_057699.2\| | solute carrier family 6 (neurotransmitter | |
| | transporter, GABA), member 13; GABA transport protein [Homo sapiens] | |
| gi\|18490233\|gb\|AAH22392.1\| | Unknown (protein for MGC: 24098) [Homo | |
| | sapiens] Length = 602 | |
| gi\|1082307\|pir\|\|JC2386 | creatine transporter BS2M - human | |
| gi\|765234\|gb\|AAB32284.1\| | creatine transporter; hCRT-BS2M [Homo sapiens] | |
| | Length = 635 | |
| gi\|13122803\|gb\|AF117999.1\|AF117999 | Homo sapiens sodium- and chloride- | |
| | dependent glycine transporter type II mRNA, complete cds Length = 2394 | |
| gi\|4759135\|ref\|NM_004211.1\| | Homo sapiens solute carrier family 6 | |
| | (neurotransmitter transporter, glycine), member 5 (SLC6A5), mRNA Length = 2729 | |
| gi\|4003524\|gb\|AF085412.1\|AF085412 | Homo sapiens glycine transporter GLYT2 | |
| | (GLYT2) mRNA, complete cds Length = 2729 | |
| gi\|4689409\|gb\|AF142501.1\|AF142501 | Homo sapiens glycine transporter type- | |
| | 2 mRNA, complete cds Length = 2450 | |
| gi\|13549153\|gb\|AF352733.1\|AF352733 | Homo sapiens glycine type 2 | |
| | transporter variant SC6 mRNA, complete cds Length = 2394 | |
| gi\|6005714\|ref\|NM_007231.1\| | Homo sapiens solute carrier family 6 | |
| | (neurotransmitter transporter), member 14 (SLC6A14), mRNA Length = 4520 | |
| gi\|5732679\|gb\|AF151978.1\|AF151978 | Homo sapiens amino acid transporter | |
| | B0+ (ATB0+) mRNA, complete cds Length = 4520 | |
| gi\|5902093\|ref\|NM_006934.1\| | Homo sapiens solute carrier family 6 | |
| | (neurotransmitter transporter, glycine), member 9 (SLC6A9), mRNA Length = 2202 | |
| gi\|546770\|gb\|S70612.1\|S70612 | glycine transporter type 1c {alternatively | |
| | spliced} [human, substantia nigra, mRNA, 2202 nt] Length = 2202 | |
| gi\|546768\|gb\|S70609.1\|S70609 | glycine transporter type 1b [human, | |
| | substantia nigra, mRNA, 2364 nt] Length = 2364 | |
| gi\|7657588\|ref\|NM_014228.1\| | Homo sapiens solute carrier family 6 | |
| | (neurotransmitter transporter, L-proline), member 7 (SLC6A7), mRNA Length = 1911 | |
| gi\|1839269\|gb\|S80071.1\|S80071 | hPROT = brain-specific L-proline transporter | |
| | [human, hippocampus, mRNA Partial, 1911 nt] Length = 1911 | |
| gi\|21756139\|dbj\|AK096607.1\| | Homo sapiens cDNA FLJ39288 fis, clone | |
| | OCBBF2012039, highly similar to SODIUM-DEPENDENT PROLINE | |
| | TRANSPORTER Length = 3738 | |
| gi\|19118376\|gb\|BM801553.1\|BM801553 | AGENCOURT_6458947 NIH_MG . . . 208 | 3e−52 |
| gi\|30781978\|emb\|BX441976.1\|BX441976 BX441976 | Homo sapiens F . . . 193 | 8e−48 |
| gi\|30613189\|emb\|BX396704.1\|BX396704 BX396704 | Homo sapiens P . . . 192 | 2e−47 |
| gi\|15344799\|gb\|BI520007.1\|BI520007 | 603071307F1 NIH_MGC_119 . . . 187 | 6e−46 |
| gi\|31043183\|emb\|AL524923.2\|AL524923 AL524923 | Homo sapiens N . . . 183 | 1e−44 |
| gi\|5439122\|gb\|AI820043.1\|AI820043 | wj78c06.x1 NCI_CGAP_Lu19 . . . 179 | 2e−43 |
| gi\|14505632\|gb\|BI087302.1\|BI087302 | 602850955F1 NIH_MGC_10 H . . . 169 | 2e−40 |
| gi\|22356937\|gb\|BQ941459.1\|BQ941459 | AGENCOURT_8741587 NIH_MG . . . 168 | 3e−40 |
| gi\|19099809\|gb\|BM770194.1\|BM770194 | K-EST0053602 S2SNU668s1 . . . 163 | 9e−39 |
| gi\|9134513\|gb\|BE261930.1\|BE261930 | 601147452F1 NIH_MGC_19 Ho . . . 160 | 6e−38 |
| gi\|9135422\|gb\|BE262420.1\|BE262420 | 601147275F1 NIH_MGC_19 Ho . . . 160 | 8e−38 |
| gi\|31066813\|emb\|AL528964.2\|AL528964 | AL528964 Homo sapiens N . . . 153 | 9e−36 |
| gi\|5589889\|gb\|AI884725.1\|AI884725 | wl83h06.x1 NCI_CGAP_Brn25 . . . 152 | 2e−35 |
| gi\|19814721\|gb\|BQ055381.1\|BQ055381 | AGENCOURT_6838271 NIH_MG . . . 144 | 5e−35 |
| gi\|30348100\|emb\|BX360891.1\|BX360891 | BX360891 Homo sapiens P . . . 150 | 1e−34 |
| gi\|21053650\|gb\|BQ378136.1\|BQ378136 | RC2-UT0021-070800-014-c1 . . . 150 | 1e−34 |
| gi\|18803583\|gb\|BM559743.1\|BM559743 | AGENCOURT_6565490 NIH_MG . . . 149 | 2e−34 |
| gi\|21120579\|gb\|BQ425264.1\|BQ425264 | AGENCOURT_7826736 NIH_MG . . . 145 | 3e−33 |
| gi\|15747978\|gb\|BI756400.1\|BI756400 | 603029207F1 NIH_MGC_114 . . . 145 | 3e−33 |
| gi\|9131468\|gb\|BE260309.1\|BE260309 | 601151167F1 NIH_MGC_19 Ho . . . 105 | 3e−33 |
| gi\|19813991\|gb\|BQ054651.1\|BQ054651 | AGENCOURT_6771313 NIH_MG . . . 145 | 3e−33 |
| gi\|11514901\|gb\|BF448732.1\|BF448732 | 7n93h01.x1 NCI_CGAP_Ov18 . . . 144 | 3e−33 |
| gi\|22702834\|gb\|BU188850.1\|BU188850 | AGENCOURT_7969087 NIH_MG . . . 143 | 1e−32 |
| gi\|19891467\|gb\|BQ063589.1\|BQ063589 | AGENCOURT_6873228 NIH_MG . . . 142 | 2e−32 |
| gi\|16200322\|gb\|BI919202.1\|BI919202 | 603177756F1 NIH_MGC_121 . . . 107 | 3e−32 |
| gi\|19100827\|gb\|BM771212.1\|BM771212 | K-EST0055038 S2SNU668s1 . . . 141 | 4e−32 |
| gi\|10991875\|dbj\|AU131521.1\|AU131521 | AU131521 NT2RP3 Homo sa . . . 140 | 6e−32 |
| gi\|24725764\|gb\|CA392750.1\|CA392750 | cs28b03.y2 Human Retinal . . . 140 | 8e−32 |
| gi\|3087130\|gb\|AA932218.1\|AA932218 | om84h08.s1 NCI_CGAP_Kid3 . . . 140 | 8e−32 |
| gi\|19029420\|gb\|BM716162.1\|BM716162 | UI-E-CI1-afw-d-22-0-UI.r . . . 139 | 1e−31 |
| gi\|21767366\|gb\|BQ643194.1\|BQ643194 | AGENCOURT_8286115 NIH_MG . . . 139 | 2e−31 |
| gi\|15753300\|gb\|BI761722.1\|BI761722 | 603046595F1 NIH_MGC_116 . . . 139 | 2e−31 |
| gi\|30625929\|emb\|BX399758.1\|BX399758 | BX399758 Homo sapiens P . . . 137 | 5e−31 |
| gi\|16177166\|gb\|BI912893.1\|BI912893 | 603176654F1 NIH_MGC_121 . . . 137 | 5e−31 |
| gi\|18999835\|gb\|BM686577.1\|BM686577 | UI-E-CQ0-ado-c-02-0-UI.r . . . 137 | 7e−31 |
| gi\|4072745\|gb\|AI335818.1\|AI335818 | qt37a10.x1 Soares_pregnan . . . 136 | 2e−30 |
| gi\|6704567\|gb\|AW297931.1\|AW297931 | UI-H-BW0-ajn-c-05-0-UI.s1 . . . 136 | 2e−30 |
| gi\|3739418\|gb\|AI188209.1\|AI188209 | qd66g05.x1 Soares_testis_. . . 136 | 2e−30 |
| gi\|12357916\|gb\|BF940596.1\|BF940596 | nae22g02.x1 NCI_CGAP_Ov1 . . . 136 | 2e−30 |
| gi\|10812049\|gb\|BF058153.1\|BF058153 | 7k21d01.x1 NCI_CGAP_Ov18 . . . 136 | 2e−30 |
| gi\|4194852\|gb\|AI382071.1\|AI382071 | te68b12.x1 Soares_NFL_T_G . . . 136 | 2e−30 |
| gi\|3739782\|gb\|AI188573.1\|AI188573 | qd15b02.x1 Soares_placent . . . 136 | 2e−30 |
| gi\|19369645\|gb\|BM919266.1\|BM919266 | AGENCOURT_6715805 NIH_MG . . . 136 | 2e−30 |
| gi\|9132691\|gb\|BE313137.1\|BE313137 | 601151680F1 NIH_MGC_19 Ho . . . 135 | 2e−30 |
| gi\|4391784\|gb\|AI499802.1\|AI499802 | tm92f12.x1 NCI_CGAP_Brn25 . . . 135 | 3e−30 |
| gi\|19816482\|gb\|BQ057142.1\|BQ057142 | AGENCOURT_6769199 NIH_MG . . . 135 | 3e−30 |
| gi\|4019101\|gb\|AI313496.1\|AI313496 | qp80g03.x1 Soares_fetal_1 . . . 135 | 3e−30 |
| gi\|10037204\|gb\|BE676663.1\|BE676663 | 7f33h12.x1 NCI_CGAP_CLL1 . . . 133 | 1e−29 |
| gi\|5659127\|gb\|AI923163.1\|AI923163 | wn67a04.x1 NCI_CGAP_Lu19 . . . 133 | 1e−29 |
| gi\|6299529\|gb\|AW160496.1\|AW160496 | au73c03.y1 Schneider feta . . . 132 | 2e−29 |
| gi\|3888138\|gb\|AI268971.1\|AI268971 | qj67e06.x1 NCI_CGAP_Kid3 . . . 131 | 4e−29 |
| gi\|24951625\|gb\|CA488834.1\|CA488834 | AGENCOURT_10808403 MAPcL . . . 131 | 5e−29 |
| gi\|19101479\|gb\|BM771864.1\|BM771864 | K-EST0055876 S2SNU668s1 . . . 130 | 8e−29 |
| gi\|19100834\|gb\|BM771219.1\|BM771219 | K-EST0055047 S2SNU668s1 . . . 130 | 8e−29 |
| gi\|30285723\|gb\|CB991203.1\|CB991203 | AGENCOURT_13627536 NIH_M . . . 129 | 1e−28 |
| gi\|6402067\|gb\|AW170542.1\|AW170542 | xn63c05.x1 Soares_NHCeC_c . . . 129 | 2e−28 |
| gi\|3770037\|gb\|AI208095.1\|AI208095 | qg51g02.x1 Soares_testis_. . . 129 | 2e−28 |
| gi\|19815947\|gb\|BQ056607.1\|BQ056607 | AGENCOURT_6792638 NIH_MG . . . 129 | 2e−28 |
| gi\|3245737\|gb\|AI028428.1\|AI028428 | ow43h03.x1 Soares_parathy . . . 128 | 4e−28 |
| gi\|2932695\|gb\|AA846555.1\|AA846555 | aj97a07.s1 Soares_parathy . . . 125 | 2e−27 |
| gi\|19027704\|gb\|BM714446.1\|BM714446 | UI-E-EJ0-ahs-b-14-0-UI.r . . . 124 | 5e−27 |
| gi\|2779557\|gb\|AA740965.1\|AA740965 | ob29g10.s1 NCI_CGAP_Kid5 . . . 122 | 3e−26 |
| gi\|15754581\|gb\|BI763003.1\|BI763003 | 603048288F1 NIH_MGC_116 . . . 87 | 4e−26 |
| gi\|15757501\|gb\|BI765923.1\|BI765923 | 603047124F1 NIH_MGC_116 . . . 97 | 6e−26 |
| gi\|20866613\|gb\|BQ311065.1\|BQ311065 | MR0-BN0070-080400-012-c0 . . . 120 | 7e−26 |
| gi\|2335442\|gb\|AA563803.1\|AA563803 | nj08h03.s1 NCI_CGAP_Pr22 . . . 83 | 1e−25 |
| gi\|14075514\|gb\|BG764861.1\|BG764861 | 602737289F1 NIH_MGC_49 H . . . 108 | 2e−25 |
| gi\|9135193\|gb\|BE262298.1\|BE262298 | 601152103F1 NIH_MGC_19 Ho . . . 112 | 2e−25 |
| gi\|28847649\|emb\|BX283195.1\|BX283195 | BX283195 NIH_MGC_99 Hom . . . 118 | 3e−25 |
| gi\|5368708\|gb\|AI803236.1\|AI803236 | tc38f07.x1 Soares_total_f . . . 117 | 1e−24 |
| gi\|5392843\|gb\|AI806277.1\|AI806277 | wf01g08.x1 Soares_NFL_T_G . . . 117 | 1e−24 |
| gi\|13337572\|gb\|BG431066.1\|BG431066 | 602498683F1 NIH_MGC_75 H . . . 112 | 1e−24 |
| gi\|15431578\|gb\|BI544266.1\|BI544266 | 603241641F1 NIH_MGC_95 H . . . 116 | 2e−24 |
| gi\|30283533\|gb\|CB989013.1\|CB989013 | AGENCOURT_13890758 NIH_M . . . 116 | 2e−24 |
| gi\|4392360\|gb\|AI500378.1\|AI500378 | tm95h12.x1 NCI_CGAP_Brn25 . . . 116 | 2e−24 |
| gi\|2397884\|gb\|AA587070.1\|AA587070 | nn77g06.s1 NCI_CGAP_Co9 H . . . 115 | 3e−24 |
| gi\|2986588\|gb\|AA877623.1\|AA877623 | nr02a08.s1 NCI_CGAP_Co10 . . . 114 | 8e−24 |
| gi\|2742945\|gb\|AA725238.1\|AA725238 | ai16a08.s1 Soares_parathy . . . 114 | 8e−24 |
| gi\|2768515\|gb\|AA737758.1\|AA737758 | nx09d11.s1 NCI_CGAP_GC3 H . . . 114 | 8e−24 |
| gi\|1155384\|gb\|N34242.1\|N34242 | yx79c08.r1 Soares melanocyte . . . 114 | 8e−24 |
| gi\|10316897\|gb\|BE868121.1\|BE868121 | 601443439F1 NIH_MGC_65 H . . . 85 | 8e−24 |
| gi\|12770164\|gb\|BG260348.1\|BG260348 | 602371470F1 NIH_MGC_93 H . . . 112 | 3e−23 |
| gi\|1110065\|gb\|H96579.1\|H96579 | yw02c10.s1 Soares melanocyte . . . 97 | 5e−23 |
| gi\|10992426\|dbj\|AU132072.1\|AU132072 | AU132072 NT2RP3 Homo sa . . . 110 | 7e−23 |
| gi\|22703431\|gb\|BU189447.1\|BU189447 | AGENCOURT_7970890 NIH_MG . . . 110 | 1e−22 |
| gi\|1138301\|gb\|N24151.1\|N24151 | yx95h11.s1 Soares melanocyte . . . 94 | 6e−22 |
| gi\|1139952\|gb\|N25604.1\|N25604 | yx77f04.s1 Soares melanocyte . . . 107 | 8e−22 |
| gi\|19373158\|gb\|BM922779.1\|BM922779 | AGENCOURT_6652753 NIH_MG . . . 85 | 2e−21 |
| gi\|14076613\|gb\|BG765960.1\|BG765960 | 602738013F1 NIH_MGC_49 H . . . 99 | 2e−21 |
| gi\|1219766\|gb\|N67641.1\|N67641 | yz94h11.s1 Soares melanocyte . . . 86 | 2e−21 |
| gi\|1123595\|gb\|H98927.1\|H98927 | yx31c10.s1 Soares melanocyte . . . 92 | 3e−21 |
| gi\|12387410\|gb\|BF984598.1\|BF984598 | 602309923F1 NIH_MGC_88 H . . . 104 | 6e−21 |
| gi\|13408756\|gb\|BG476477.1\|BG476477 | 602522011F1 NIH_MGC_20 H . . . 103 | 1e−20 |
| gi\|30288988\|gb\|CB994468.1\|CB994468 | AGENCOURT_13671717 NIH_M . . . 87 | 1e−20 |
| gi\|21041481\|gb\|BQ365969.1\|BQ365969 | QV4-GN0120-250900-420-a0 . . . 103 | 1e−20 |
| gi\|14081606\|gb\|BG770953.1\|BG770953 | 602719177F1 NIH_MGC_60 H . . . 90 | 2e−20 |
| gi\|2994061\|gb\|AA884531.1\|AA884531 | aj61f09.s1 Soares_testis_. . . 102 | 2e−20 |
| gi\|10991548\|dbj\|AU131194.1\|AU131194 | AU131194 NT2RP3 Homo sa . . . 100 | 7e−20 |
| gi\|15584192\|gb\|BI669959.1\|BI669959 | 603294467F1 NIH_MGC_96 H . . . 100 | 1e−19 |
| gi\|19373587\|gb\|BM923208.1\|BM923208 | AGENCOURT_6626009 NIH_MG . . . 100 | 1e−19 |

The acetylcholine transporters of this invention are useful in a number of contexts. For example, they provide good targets to screen for agents that modulate (e.g. upregulate) acetylcholine transporter expression and/or activity and thereby regulate acetylcholine transport and consequently activity of cholinergic synapses. They also provide a good target for agents to modulate cholinergic synapse activity and thereby mitigate one or more symptoms of a pathology characterized by abnormal cholinergic synapse activity.

I. Assays for Modulators of Acetylcholine Expression and/or Activity.

As indicated above, in one aspect, this invention is premised, in part, on the discovery acetylcholine transporters. It is believed that activity these transporters are critical for healthy neurological activity and upregulation of such receptors can mitigate adverse effects of a variety of neuropathologies (e.g. ALS, epilepsy, Parkinsons disease, Alzheimer's disease, etc.). Conversely, inhibition of the acetylcholine transporters can have beneficial effects in certain circumstances.

Thus, in certain embodiments, this invention provides methods of screening for agents that modulate expression and/or activity of acetylcholine transporters (i.e., the acetylcholine transporters and/or orthologues identified herein). In certain embodiments, the methods involve contacting a cell comprising a acetylcholine transporter nucleic acid (e.g. the C. elegans acetylcholine transporter and/or orthologues thereof identified herein) with a test agent; and detecting the expression or activity of the acetylcholine transporter(s) wherein a difference in the expression of the acetylcholine transporter(s) of the cell as compared to the activity the acetylcholine transporter(s) of a control cell (e.g. a cell of the same type that is contacted with a lower concentration of test agent or no test agent) indicates that the test agent alters acetylcholine transporter expression and/or activity.

Detection of changes in metabolic activity can involve detecting the expression level and/or activity level of acetylcholine transporter genes or gene products or acetylcholine transporter polypeptides or polypeptide activity.

Expression levels of a gene can be altered by changes in the transcription of the gene product (i.e. transcription of mRNA), and/or by changes in translation of the gene product (i.e. translation of the protein), and/or by post-translational modification(s) (e.g. protein folding, glycosylation, etc.). Thus preferred assays of this invention include assaying for level of transcribed mRNA (or other nucleic acids derived from the subject genes), level of translated protein, activity of translated protein, etc. Examples of such approaches are described below.

A) Nucleic-Acid Based Assays.

1) Target Molecules.

Changes in expression level can be detected by measuring changes in genomic DNA or a nucleic acid derived from the genomic DNA (e.g. the acetylcholine transporters and/or orthologues identified herein). In order to measure the expression level it is desirable to provide a nucleic acid sample for such analysis. In preferred embodiments the nucleic acid is found in or derived from a biological sample. The term “biological sample”, as used herein, refers to a sample obtained from an organism or from components (e.g., cells) of an organism. The sample may be of any biological tissue or fluid. Biological samples may also include organs or sections of tissues such as frozen sections taken for histological purposes. Biological samples also include cells in culture and the cells can be native cells or recombinantly modified cells (e.g. modified to express a heterologous acetylcholine transporter).

The nucleic acid (e.g., acetylcholine transporter mRNA or a nucleic acid derived from a acetylcholine transporter mRNA) is, in certain preferred embodiments, isolated from the sample according to any of a number of methods well known to those of skill in the art. Methods of isolating mRNA are well known to those of skill in the art. For example, methods of isolation and purification of nucleic acids are described in detail in by Tijssen ed., (1993) Chapter 3 of Laboratory Techniques in Biochemistry and Molecular Biology: Hybridization With Nucleic Acid Probes, Part I. Theory and Nucleic Acid Preparation, Elsevier, N.Y. and Tijssen ed.

In a preferred embodiment, the “total” nucleic acid is isolated from a given sample using, for example, an acid guanidinium-phenol-chloroform extraction method and polyA+ mRNA is isolated by oligo dT column chromatography or by using (dT)ₙ magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. (1987) Greene Publishing and Wiley-Interscience, New York).

Frequently, it is desirable to amplify the nucleic acid sample prior to assaying for expression level. Methods of amplifying nucleic acids are well known to those of skill in the art and include, but are not limited to polymerase chain reaction (PCR, see. e.g., Innis, et al., (1990) PCR Protocols. A guide to Methods and Application. Academic Press, Inc. San Diego), ligase chain reaction (LCR) (see Wu and Wallace (1989) Genomics 4: 560, Landegren et al. (1988) Science 241: 1077, and Barringer et al. (1990) Gene 89: 117, transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.).

In a particularly preferred embodiment, where it is desired to quantify the transcription level (and thereby expression) (e.g. of an acetylcholine transporter) in a sample, the nucleic acid sample is one in which the concentration of the acetylcholine transporter mRNA transcript(s), or the concentration of the nucleic acids derived from the mRNA transcript(s), is proportional to the transcription level (and therefore expression level) of that gene. Similarly, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Thus, for example, an assay where a 5 fold difference in concentration of the target mRNA results in a 3 to 6 fold difference in hybridization intensity is sufficient for most purposes.

Where more precise quantification is required appropriate controls can be run to correct for variations introduced in sample preparation and hybridization as described herein. In addition, serial dilutions of “standard” target nucleic acids (e.g., mRNAs) can be used to prepare calibration curves according to methods well known to those of skill in the art. Of course, where simple detection of the presence or absence of a transcript or large changes in nucleic acid concentration are desired, no elaborate control or calibration is required.

In the simplest embodiment, the sample nucleic acid sample is the total mRNA or a total cDNA isolated and/or otherwise derived from a biological sample. The nucleic acid may be isolated from the sample according to any of a number of methods well known to those of skill in the art as indicated above.

2) Hybridization-Based Assays.

The expression of particular genes (e.g. the C. elegans acetylcholine transporters and/or orthologues identified herein) can be routinely detected and/or quantitated using nucleic acid hybridization techniques (see, e.g., Sambrook et al. supra). For example, one method for evaluating the presence, absence, or quantity of a particular genomic DNA or reverse-transcribed cDNA involves a “Southern Blot”. In a Southern Blot, the DNA sample is typically fragmented and separated on an electrophoretic gel and hybridized to a probe specific for the nucleic acid(s) of interest. Comparison of the intensity of the hybridization signal from the probe with a “control” probe (e.g. a probe for a “housekeeping gene) provides an estimate of the relative expression level of the target nucleic acid (e.g. a ACETYLCHOLINE nucleic acid).

Alternatively, the acetylcholine transporter mRNA can be directly quantified in a Northern blot. In brief, the mRNA is isolated from a given cell sample using, for example, an acid guanidinium-phenol-chloroform extraction method. The mRNA is then electrophoresed to separate the mRNA species and the mRNA is then transferred from the gel to a membrane (e.g. a nitrocellulose membrane). As with the Southern blots, labeled probes are used to identify and/or quantify the target (acetylcholine transporter) mRNA. Appropriate controls (e.g. probes to housekeeping genes) provide a reference for evaluating relative acetylcholine transporter expression level.

An alternative means for determining the particular nucleic acid expression levels is in situ hybridization. In situ hybridization assays are well known (e.g., Angerer (1987) Meth. Enzymol 152: 649). Generally, in situ hybridization comprises the following major steps: (1) fixation of tissue or biological structure to be analyzed; (2) prehybridization treatment of the biological structure to increase accessibility of target DNA, and to reduce nonspecific binding; (3) hybridization of the mixture of nucleic acids to the nucleic acid in the biological structure or tissue; (4) post-hybridization washes to remove nucleic acid fragments not bound in the hybridization and (5) detection of the hybridized nucleic acid fragments. The reagent used in each of these steps and the conditions for use vary depending on the particular application.

In some applications it is necessary to block the hybridization capacity of repetitive sequences. Thus, in some embodiments, tRNA, human genomic DNA, or Cot-1 DNA is used to block non-specific hybridization.

3) Amplification-Based Assays.

In another embodiment, amplification-based assays can be used to measure expression (transcription) level of particular genes (e.g. the C. elegans acetylcholine transporters and/or orthologues identified herein). In such amplification-based assays, the target nucleic acid sequences act as template(s) in amplification reaction(s) (e.g. Polymerase Chain Reaction (PCR) or reverse-transcription PCR (RT-PCR)). In a quantitative amplification, the amount of amplification product will be proportional to the amount of template in the original sample. Comparison to appropriate controls (e.g. tissue or cells exposed to the test agent at a different concentration or not exposed to the test agent) provides a measure of the target transcript level.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). One approach, for example, involves simultaneously co-amplifying a known quantity of a control sequence using the same primers as those used to amplify the target. This provides an internal standard that may be used to calibrate the PCR reaction.

4) Hybridization Formats and Optimization of hybridization conditions.

a) Array-Based Hybridization Formats.

In one embodiment, the methods of this invention can be utilized in array-based hybridization formats. Arrays are a multiplicity of different “probe” or “target” nucleic acids (or other compounds) attached to one or more surfaces (e.g., solid, membrane, or gel). In a preferred embodiment, the multiplicity of nucleic acids (or other moieties) is attached to a single contiguous surface or to a multiplicity of surfaces juxtaposed to each other.

In an array format a large number of different hybridization reactions can be run essentially “in parallel.” This provides rapid, essentially simultaneous, evaluation of a number of hybridizations in a single “experiment”. Methods of performing hybridization reactions in array-based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays can be produced according to a wide variety of methods well known to those of skill in the art. For example, in a simple embodiment, “low density” arrays can simply be produced by spotting (e.g. by hand using a pipette) different nucleic acids at different locations on a solid support (e.g. a glass surface, a membrane, etc.).

This simple spotting, approach has been automated to produce high density spotted arrays (see, e.g., U.S. Pat. No. 5,807,522). This patent describes the use of an automated system that taps a microcapillary against a surface to deposit a small volume of a biological sample. The process is repeated to generate high-density arrays.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide arrays. Synthesis of high density arrays is also described in U.S. Pat. Nos. 5,744,305, 5,800,992 and 5,445,934.

b) Other Hybridization Formats.

A wide variety of nucleic acid hybridization formats are known to those skilled in the art. For example, common formats include sandwich assays and competition or displacement assays. Such assay formats are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587.

Sandwich assays are commercially useful hybridization assays for detecting or isolating nucleic acid sequences. Such assays utilize a “capture” nucleic acid covalently immobilized to a solid support and a labeled “signal” nucleic acid in solution. The sample will provide the target nucleic acid. The “capture” nucleic acid and “signal” nucleic acid probe hybridize with the target nucleic acid to form a “sandwich” hybridization complex. To be most effective, the signal nucleic acid should not hybridize with the capture nucleic acid.

Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P-labelled probes or the like. Other labels include ligands that bind to labeled antibodies, fluorophores, chemiluminescent agents, enzymes, and antibodies which can serve as specific binding pair members for a labeled ligand.

Detection of a hybridization complex may involve the binding of a signal generating complex to a duplex of target and probe polynucleotides or nucleic acids. Typically, such binding occurs through ligand and anti-ligand interactions as between a ligand-conjugated probe and an anti-ligand conjugated with a signal.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence based amplification (NASBAO, Cangene, Mississauga, Ontario) and Q Beta Replicase systems.

c) Optimization of Hybridization Conditions.

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids, or in the addition of chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency to ensure hybridization and then subsequent washes are performed at higher stringency to eliminate mismatched hybrid duplexes. Successive washes may be performed at increasingly higher stringency (e.g., down to as low as 0.25×SSPE at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be present.

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. Thus, in a preferred embodiment, the hybridized array may be washed at successively higher stringency solutions and read between each wash. Analysis of the data sets thus produced will reveal a wash stringency above which the hybridization pattern is not appreciably altered and which provides adequate signal for the particular probes of interest.

In a preferred embodiment, background signal is reduced by the use of a blocking reagent (e.g., tRNA, sperm DNA, cot-1 DNA, etc.) during the hybridization to reduce non-specific binding. The use of blocking agents in hybridization is well known to those of skill in the art (see, e.g., Chapter 8 in P. Tijssen, supra.).

Methods of optimizing hybridization conditions are well known to those of skill in the art (see, e.g., Tijssen (1993) Laboratory Techniques in Biochemistry and Molecular Biology, Vol. 24: Hybridization With Nucleic Acid Probes, Elsevier, N.Y.).

Optimal conditions are also a function of the sensitivity of label (e.g., fluorescence) detection for different combinations of substrate type, fluorochrome, excitation and emission bands, spot size and the like. Low fluorescence background surfaces can be used (see, e.g., Chu (1992) Electrophoresis 13:105-114). The sensitivity for detection of spots (“target elements”) of various diameters on the candidate surfaces can be readily determined by, e.g., spotting a dilution series of fluorescently end labeled DNA fragments. These spots are then imaged using conventional fluorescence microscopy. The sensitivity, linearity, and dynamic range achievable from the various combinations of fluorochrome and solid surfaces (e.g., glass, fused silica, etc.) can thus be determined. Serial dilutions of pairs of fluorochrome in known relative proportions can also be analyzed. This determines the accuracy with which fluorescence ratio measurements reflect actual fluorochrome ratios over the dynamic range permitted by the detectors and fluorescence of the substrate upon which the probe has been fixed.

d) Labeling and Detection of Nucleic Acids.

The probes used herein for detection of acetylcholine transporter expression levels can be full length or less than the full length of the acetylcholine transporter of interest (e.g. the C. elegans acetylcholine transporters and/or orthologues identified herein) mRNA. Shorter probes are empirically tested for specificity. Preferred probes are sufficiently long so as to specifically hybridize with the acetylcholine transporter target nucleic acid(s) under stringent conditions. The preferred size range is from about 20 bases to the length of the acetylcholine transporter mRNA, more preferably from about 30 bases to the length of the acetylcholine transporter mRNA, and most preferably from about 40 bases to the length of the acetylcholine transporter mRNA.

The probes are typically labeled, with a detectable label. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, or ³²P), enzymes (e.g., horse radish peroxidase, alkaline phosphatase and others commonly used in an ELISA), and calorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

A fluorescent label is preferred because it provides a very strong signal with low background. It is also optically detectable at high resolution and sensitivity through a quick scanning procedure. The nucleic acid samples can all be labeled with a single label, e.g., a single fluorescent label. Alternatively, in another embodiment, different nucleic acid samples can be simultaneously hybridized where each nucleic acid sample has a different label. For instance, one target could have a green fluorescent label and a second target could have a red fluorescent label. The scanning step will distinguish sites of binding of the red label from those binding the green fluorescent label. Each nucleic acid sample (target nucleic acid) can be analyzed independently from one another.

Suitable chromogens which can be employed include those molecules and compounds which absorb light in a distinctive range of wavelengths so that a color can be observed or, alternatively, that emit light when irradiated with radiation of a particular wave length or wave length range, e.g., fluorescent molecules.

Desirably, fluorescent labels should absorb light above about 300 nm, preferably about 350 nm, and more preferably above about 400 nm, usually emitting at wavelengths greater than about 10 nm higher than the wavelength of the light absorbed.

Detectable signal can also be provided by chemiluminescent and bioluminescent sources. Chemiluminescent sources include compounds that become electronically excited by a chemical reaction and can then emit light that serves as the detectable signal or donates energy to a fluorescent acceptor. Alternatively, luciferins can be used in conjunction with luciferase or lucigenins to provide bioluminescence.

Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

The label can be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

Fluorescent labels are easily added during an in vitro transcription reaction. Thus, for example, fluorescein labeled UTP and CTP can be incorporated into the RNA produced in an in vitro transcription.

The labels can be attached directly or through a linker moiety. In general, the site of label or linker-label attachment is not limited to any specific position. For example, a label may be attached to a nucleoside, nucleotide, or analogue thereof at any position that does not interfere with detection or hybridization as desired. For example, certain Label-On Reagents from Clontech (Palo Alto, Calif.) provide for labeling interspersed throughout the phosphate backbone of an oligonucleotide and for terminal labeling at the 3′ and 5′ ends. As shown for example herein, labels can be attached at positions on the ribose ring or the ribose can be modified and even eliminated as desired. The base moieties of useful labeling reagents can include those that are naturally occurring or modified in a manner that does not interfere with the purpose to which they are put. Modified bases include but are not limited to 7-deaza A and G, 7-deaza-8-aza A and G, and other heterocyclic moieties.

It will be recognized that fluorescent labels are not to be limited to single species of organic molecules, but include inorganic molecules, multi-molecular mixtures of organic and/or inorganic molecules, crystals, heteropolymers, and the like. Thus, for example, CdSe-CdS core-shell nanocrystals enclosed in a silica shell can be easily derivatized for coupling to a biological molecule (Bruchez et al. (1998) Science, 281: 2013-2016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018).

B) Acetylcholine Transporter Polypeptide-Based Assays—Polypeptide Expression.

1) Assay Formats.

In addition to, or in alternative to, the detection of nucleic acid expression level(s), alterations in expression of acetylcholine transporters can be detected and/or quantified by detecting and/or quantifying the amount and/or activity of translated acetylcholine transporter polypeptide or fragments thereof.

2) Detection of Expressed Protein.

The acetylcholine transporter polypeptides to be assayed can be detected and quantified by any of a number of methods well known to those of skill in the art. These include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, western blotting, and the like.

In one preferred embodiment, the acetylcholine transporter polypeptide(s) are detected/quantified in an electrophoretic protein separation (e.g. a 1- or 2-dimensional electrophoresis). Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In another preferred embodiment, Western blot (immunoblot) analysis is used to detect and quantify the presence of polypeptide(s) of this invention in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the target polypeptide(s).

The antibodies specifically bind to the target acetylcholine transporter polypeptide(s) and can be directly labeled or alternatively may be subsequently detected using labeled antibodies (e.g., labeled sheep anti-mouse antibodies) that specifically bind to the a domain of the antibody.

In preferred embodiments, the acetylcholine transporter polypeptide(s) (e.g. C. elegans acetylcholine transporter and/or the homologues or orthologues thereof identified herein) are detected using an immunoassay. As used herein, an immunoassay is an assay that utilizes an antibody to specifically bind to the analyte (e.g., the target polypeptide(s)). The immunoassay is thus characterized by detection of specific binding of a polypeptide of this invention to an antibody as opposed to the use of other physical or chemical properties to isolate, target, and quantify the analyte.

Any of a number of well recognized immunological binding assays (see, e.g., U.S. Pat. Nos. 4,366,241; 4,376,110; 4,517,288; and 4,837,168) are well suited to detection or quantification of the polypeptide(s) identified herein. For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte. In preferred embodiments, the capture agent is an antibody.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent/polypeptide complex.

Other proteins capable of specifically binding immunoglobulin constant regions, such as protein A or protein G may also be used as the label agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

Preferred immunoassays for detecting the target polypeptide(s) are either competitive or noncompetitive. Noncompetitive immunoassays are assays in which the amount of captured analyte is directly measured. In one preferred “sandwich” assay, for example, the capture agents (antibodies) can be bound directly to a solid substrate where they are immobilized. These immobilized antibodies then capture the target polypeptide present in the test sample. The target polypeptide thus immobilized is then bound by a labeling agent, such as a second antibody bearing a label.

In competitive assays, the amount of analyte (e.g. acetylcholine transporter) present in the sample is measured indirectly by measuring the amount of an added (exogenous) analyte displaced (or competed away) from a capture agent (antibody) by the analyte present in the sample. For example, in one competitive assay, a known amount of, labeled acetylcholine transporter polypeptide is added to the sample and the sample is then contacted with a capture agent. The amount of labeled polypeptide bound to the antibody is inversely proportional to the concentration of target polypeptide present in the sample.

In one particularly preferred embodiment, the antibody is immobilized on a solid substrate. The amount of target polypeptide bound to the antibody may be determined either by measuring the amount of target polypeptide present in a polypeptide/antibody complex, or alternatively by measuring the amount of remaining uncomplexed polypeptide.

The immunoassay methods of the present invention include an enzyme immunoassay (EIA) which utilizes, depending on the particular protocol employed, unlabeled or labeled (e.g., enzyme-labeled) derivatives of polyclonal or monoclonal antibodies or antibody fragments or single-chain antibodies. In certain embodiments the antibodies are antibodies that bind to an acetylcholine transporter polypeptide. Any of the known modifications of EIA, for example, enzyme-linked immunoabsorbent assay (ELISA), may also be employed. As indicated above, also contemplated by the present invention are immunoblotting immunoassay techniques such as western blotting employing an enzymatic detection system.

The immunoassay methods of the present invention may also include other known immunoassay methods, for example, fluorescent immunoassays using antibody conjugates or antigen conjugates of fluorescent substances such as fluorescein or rhodamine, latex agglutination with antibody-coated or antigen-coated latex particles, haemagglutination with antibody-coated or antigen-coated red blood corpuscles, and immunoassays employing an avidin-biotin or streptavidin-biotin detection systems, and the like.

The particular parameters employed in the immunoassays of the present invention can vary widely depending on various factors such as the concentration of antigen in the sample, the nature of the sample, the type of immunoassay employed and the like. Optimal conditions can be readily established by those of ordinary skill in the art. In certain embodiments, the amount of antibody that binds the acetylcholine transporter polypeptide is typically selected to give 50% binding of detectable marker in the absence of sample. If purified antibody is used as the antibody source, the amount of antibody used per assay will generally range from about 1 ng to about 100 ng. Typical assay conditions include a temperature range of about 4° C. to about 45° C., preferably about 25° C. to about 37° C., and most preferably about 25° C., a pH value range of about 5 to 9, preferably about 7, and an ionic strength varying from that of distilled water to that of about 0.2M sodium chloride, preferably about that of 0.15M sodium chloride. Times will vary widely depending upon the nature of the assay, and generally range from about 0.1 minute to about 24 hours. A wide variety of buffers, for example PBS, may be employed, and other reagents such as salt to enhance ionic strength, proteins such as serum albumins, stabilizers, biocides and non-ionic detergents may also be included.

The assays of this invention are scored (as positive or negative or quantity of target C. acetylcholine transporter polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

Antibodies for use in the various immunoassays described herein, are commercially available or can be produced as described below.

3) Antibodies to Acetylcholine Transporter Polypeptides.

Polyclonal antibodies, monoclonal antibodies, single chain antibodies, and the like (e.g., anti-acetylcholine transporter antibodies) can be used in the immunoassays of the invention described herein. Polyclonal antibodies are preferably raised by multiple injections (e.g. subcutaneous or intramuscular injections) of substantially pure polypeptides (e.g. C. elegans acetylcholine transporter and/or the homologues or orthologues thereof or fragments thereof) into a suitable non-human mammal. The antigenicity of the target peptides can be determined by conventional techniques to determine the magnitude of the antibody response of an animal that has been immunized with the peptide. Generally, the peptides that are used to raise antibodies for use in the methods of this invention should generally be those that induce production of high titers of antibody with relatively high affinity for target polypeptide.

If desired, the immunizing acetylcholine transporter peptide can be coupled to a carrier protein, e.g., by conjugation using techniques that are well-known in the art. Commonly used carriers that can be chemically coupled to the peptide include keyhole limpet hemocyanin (KLH), thyroglobulin, bovine serum albumin (BSA), tetanus toxoid, and the like. The coupled peptide is used to immunize the animal (e.g. a mouse or a rabbit).

The antibodies are then obtained from blood samples taken from the mammal. The techniques used to develop polyclonal antibodies are known in the art (see, e.g., Methods of Enzymology, “Production of Antisera With Small Doses of Immunogen: Multiple Intradermal Injections”, Langone, et al. eds. (Acad. Press, 1981)). Polyclonal antibodies produced by the animals can be further purified, for example, by binding to and elution from a matrix to which the peptide to which the antibodies were raised is bound. Those of skill in the art will know of various techniques common in the immunology arts for purification and/or concentration of polyclonal antibodies, as well as monoclonal antibodies see, for example, Coligan, et al. (1991) Unit 9, Current Protocols in Immunology, Wiley Interscience).

Preferably, however, the anti-acetylcholine transorter antibodies produced are monoclonal antibodies (“mAb's”). For preparation of monoclonal antibodies, immunization of a mouse or rat is preferred. The term “antibody” as used in this invention includes intact molecules as well as fragments thereof, such as, Fab and F(ab′)²′, and/or single-chain antibodies (e.g. scFv) that are capable of binding an epitopic determinant.

The general method used for production of hybridomas secreting mAbs is well known (Kohler and Milstein (1975) Nature, 256:495). Briefly, as described by Kohler and Milstein the technique comprises fusing an antibody-secreting cell (e.g. a splenocyte) with an immortalized cell (e.g. a myeloma cell). Hybridomas are then screened for production of antibodies that bind to an acetylcholine transporter polypeptide or a fragment thereof. Confirmation of specificity among mAb's can be accomplished using relatively routine screening techniques (such as the enzyme-linked immunosorbent assay, or “ELISA”, BiaCore, etc.) to determine the binding specificity and/or avidity of the mAb of interest.

Antibodies fragments, e.g. single chain antibodies (scFv or others), can also be produced/selected using phage display technology. The ability to express antibody fragments on the surface of viruses that infect bacteria (bacteriophage or phage) makes it possible to isolate a single binding antibody fragment, e.g., from a library of greater than 10¹⁰ nonbinding clones. To express antibody fragments on the surface of phage (phage display), an antibody fragment gene is inserted into the gene encoding a phage surface protein (e.g., pIII) and the antibody fragment-pIII fusion protein is displayed on the phage surface (McCafferty et al. (1990) Nature, 348: 552-554; Hoogenboom et al. (1991) Nucleic Acids Res. 19: 4133-4137).

Since the antibody fragments on the surface of the phage are functional, phage bearing antigen binding antibody fragments can be separated from non-binding phage by antigen affinity chromatography (McCafferty et al. (1990) Nature, 348: 552-554). Depending on the affinity of the antibody fragment, enrichment factors of 20 fold-1,000,000 fold are obtained for a single round of affinity selection. By infecting bacteria with the eluted phage, however, more phage can be grown and subjected to another round of selection. In this way, an enrichment of 1000 fold in one round can become 1,000,000 fold in two rounds of selection (McCafferty et al. (1990) Nature, 348: 552-554). Thus even when enrichments are low (Marks et al. (1991) J. Mol. Biol. 222: 581-597), multiple rounds of affinity selection can lead to the isolation of rare phage. Since selection of the phage antibody library on antigen results in enrichment, the majority of clones bind antigen after as few as three to four rounds of selection. Thus only a relatively small number of clones (several hundred) need to be analyzed for binding to antigen.

Human antibodies can be produced without prior immunization by displaying very large and diverse V-gene repertoires on phage (Marks et al. (1991) J. Mol. Biol. 222: 581-597). In one embodiment natural V_{H} and V_{L} repertoires present in human peripheral blood lymphocytes are were isolated from unimmunized donors by PCR. The V-gene repertoires were spliced together at random using PCR to create a scFv gene repertoire which is was cloned into a phage vector to create a library of 30 million phage antibodies (Id.). From this single “naive” phage antibody library, binding antibody fragments have been isolated against more than 17 different antigens, including haptens, polysaccharides and proteins (Marks et al. (1991) J. Mol. Biol. 222: 581-597; Marks et al. (1993). Bio/Technology. 10: 779-783; Griffiths et al. (1993) EMBO J. 12: 725-734; Clackson et al. (1991) Nature. 352: 624-628). Antibodies have been produced against self proteins, including human thyroglobulin, immunoglobulin, tumor necrosis factor and CEA (Griffiths et al. (1993) EMBO J. 12: 725-734). It is also possible to isolate antibodies against cell surface antigens by selecting directly on intact cells. The antibody fragments are highly specific for the antigen used for selection and have affinities in the 1:M to 100 nM range (Marks et al. (1991) J. Mol. Biol. 222: 581-597; Griffiths et al. (1993) EMBO J. 12: 725-734). Larger phage antibody libraries result in the isolation of more antibodies of higher binding affinity to a greater proportion of antigens.

It will also be recognized that antibodies can be prepared by any of a number of commercial services (e.g., Berkeley antibody laboratories, Bethyl Laboratories, Anawa, Eurogenetec, etc.).

C) Polypeptide-Based Assays—Polypeptide Activity.

In addition to, or as an alternative to, the assays described above, it is also possible to assay for acetylcholine transporter activity. Thus, acetylcholine transporter activity in a cell can be readily measured by providing a suitable ligand (e.g. labeled acetylcholine) and measuring the acetylcholine transporter-mediated uptake of the ligand.

Having identified acetylcholine transporter, methods of transfecting cells with a nucleic acid that encodes a functional acetylcholine transporter, can be routinely accomplished. Preferred cells are cells that do not normally express the acetylcholine transporter whose activity is to be assayed. Such cells include, but are not limited to oocytes (e.g., Xenopus laevis oocytes).

D) Pre-Screening for Agents that Bind Acetylcholine Transporter Nucleic Acids or Polypeptides.

In certain embodiments it is desired to pre-screen test agents for the ability to interact with (e.g. specifically bind to) a acetylcholine transporter nucleic acid or polypeptide. Specifically, binding test agents are more likely to interact with and thereby modulate acetylcholine transporter expression and/or activity. Thus, in some preferred embodiments, the test agent(s) are pre-screened for binding acetylcholine transporter nucleic acids or to acetylcholine transportera before performing the more complex assays described above.

In one embodiment, such pre-screening is accomplished with simple binding assays. Means of assaying for specific binding or the binding affinity of a particular ligand for a nucleic acid or for a protein are well known to those of skill in the art. In preferred binding assays, the acetylcholine transporter protein or protein fragment, or nucleic acid is immobilized and exposed to a test agent (which can be labeled), or alternatively, the test agent(s) are immobilized and exposed to a acetylcholine transporter polypeptide (or fragment) or to a acetylcholine transporter nucleic acid or fragment thereof (which can be labeled). The immobilized moiety is then washed to remove any unbound material and the bound test agent or bound acetylcholine transporter nucleic acid or protein is detected (e.g. by detection of a label attached to the bound molecule). The amount of immobilized label is proportional to the degree of binding between the acetylcholine transporter protein or nucleic acid and the test agent.

II. Modulator Databases.

In certain embodiments, the agents that score positively in the assays described herein (e.g. show an ability to modulate acetylcholine transporter expression or activity) can be entered into a database of putative and/or actual modulators of acetylcholine transport. The term database refers to a means for recording and retrieving information. In preferred embodiments the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

III. High Throughput Screening for Agents that Modulate Acetylcholine Transporter Expression and/or Activity.

The assays for modulators of acetylcholine transporter expression and/or activity or acetylcholine transporter ligands are also amenable to “high-throughput” modalities. Conventionally, new chemical entities with useful properties (e.g., modulation of acetylcholine transporter activity and/or expression) are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one preferred embodiment, high throughput screening methods involve providing a library containing a large number of compounds (candidate compounds) potentially having the desired activity. Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used directly in the desired application.

A) Combinatorial Chemical Libraries for Modulators of acetylcholine Transporter Expression or Activity.

The likelihood of an assay identifying an agent that modulates acetylcholine transporter activity and/or expression is increased when the number and types of test agents used in the screening system is increased. Recently, attention has focused on the use of combinatorial chemical libraries to assist in the generation of new chemical compound leads. A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9): 1233-1250).

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, 26 Dec. 1991), encoded peptides (PCT Publication WO 93/20242, 14 Oct. 1993), random bio-oligomers (PCT Publication WO 92/00091, 9 Jan. 1992), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658). See, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083) antibody libraries (see, e.g., Vaughn et al. (1996) Nature Biotechnology, 14(3): 309-314), and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, January 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines U.S. Pat. No. 5,288,514, and the like).

Methods for the synthesis of molecular libraries are well known in the art (see, for example, DeWitt et al. (1993) Proc. Natl. Acad. Sci. U.S.A. 90: 6909; Erb et al. (1994) Proc. Natl. Acad. Sci. U.S.A. 91:11422; Zuckermann et al. (1994) J. Med. Chem. 37: 2678; Cho et al. (1993) Science, 261: 1303; Carell et al. (1994) Angew. Chem. Int. Ed. Engl. 33: 2059; Gallop et al. (1994) J. Med. Chem. 37: 1233, and the like). Libraries of compounds can be presented in solution (see, e.g., Houghten (1992) Biotechniques 13: 412-421), or on solid supports including but not limited to beads (Lam (1991) Nature, 354:82-84), chips (Fodor (1993) Nature, 364: 555-556), bacteria or spores (U.S. Pat. No. 5,223,409), plasmids (Cull et al. (1992) Proc. Natl. Acad. Sci. U.S.A. 89: 1865-1869), phage (Scott & Smith (1990) Science 249: 386-390, 1990; Devlin (1990) Science 249: 404-406); Cwirla et al. (1990) Proc. Natl. Acad. Sci. 97: 6378-6382; Felici (1991) J. Mol. Biol. 222: 301-310; and U.S. Pat. No. 5,223,409).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

B) High Throughput Assays of Chemical Libraries for Modulators of Acetylcholine Transporter Expression and/or Activity.

Any of the assays for agents that modulate acetylcholine transporter expression or activity are amenable to high throughput screening. As described above likely modulators either inhibit expression of the gene product, or inhibit the activity of the receptor. Preferred assays thus detect inhibition of transcription (i.e., inhibition of mRNA production) by the test compound(s), inhibition of protein expression by the test compound(s), binding to the gene (e.g., gDNA, or cDNA) or gene product (e.g., mRNA or expressed protein) by the test compound(s). High throughput assays for the presence, absence, or quantification of particular nucleic acids or protein products are well known to those of skill in the art. Similarly, binding assays are similarly well known. Thus, for example, U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols the various high throughput. Thus, for example, Zymark Corp. provides technical bulletins describing screening systems for detecting the modulation of gene transcription, ligand binding, and the like.

IV. Providing Cells that Transport Acetylcholine.

Certain embodiments of this invention provide cells that are modified to alter their acetylcholine transporter activity. Such cells can include cells that have no endogenous acetylcholine transporter activity, or cells that have normally comprise acetylcholine transporters.

In certain embodiments the cells are convenient for assaying for acetylcholine transporter activity. In other embodiments, the cells are modified to increase acetylcholine transporter activity to treat or mitigate a pathological state. Thus, for example, where a subject (e.g. human or non-human mammal) suffers from an affliction associated with depressed acetylcholine transporter activity (e.g. ALS, Alzheimers disease, Parkinson's disease, etc.), cells in the organism can be transfected with a nucleic acid expressing a one or more heterologous ACETYLCHOLINE transporter(s) thereby increasing the ability of the cell to transport acetylcholine (e.g. into synaptic vesicles).

Methods of transiently or stably expressing heterologous nucleic acids in cells are well known to those of skill in the art. Using the sequence information provided herein and in publicly available databases, DNA encoding the acetylcholine transporter proteins described herein can be prepared by any suitable method as described above, including, for example, cloning and restriction of appropriate sequences or direct chemical synthesis by methods such as the phosphotriester method of Narang et al. (1979) Meth. Enzymol. 68: 90-99; the phosphodiester method of Brown et al. (1979) Meth. Enzymol. 68: 109-151; the diethylphosphoramidite method of Beaucage et al. (1981) Tetra. Lett., 22: 1859-1862; and the solid support method of U.S. Pat. No. 4,458,066.

Chemical synthesis produces a single stranded oligonucleotide. This may be converted into double stranded DNA by hybridization with a complementary sequence, or by polymerization with a DNA polymerase using the single strand as a template. One of skill would recognize that while chemical synthesis of DNA is limited to sequences of about 100 bases, longer sequences may be obtained by the ligation of shorter sequences.

Alternatively, subsequences may be cloned and the appropriate subsequences cleaved using appropriate restriction enzymes. The fragments may then be ligated to produce the desired DNA sequence.

In one embodiment, the acetylcholine transporter nucleic acids of this invention can be cloned using DNA amplification methods such as polymerase chain reaction (PCR) (see, e.g., Example 2). Thus, for example, the nucleic acid sequence or subsequence is PCR amplified, using a sense primer containing one restriction site (e.g., NdeI) and an antisense primer containing another restriction site (e.g., HindIII). This will produce a nucleic acid encoding the desired acetylcholine transporter sequence or subsequence and having terminal restriction sites. This nucleic acid can then be easily ligated into a vector containing a nucleic acid encoding the second molecule and having the appropriate corresponding restriction sites. Suitable PCR primers can be determined by one of skill in the art using the sequence information provided herein. Appropriate restriction sites can also be added to the nucleic acid encoding the acetylcholine transporter protein or protein subsequence by site-directed mutagenesis. The plasmid containing the acetylcholine transporter sequence or subsequence is cleaved with the appropriate restriction endonuclease and then ligated into the vector encoding the second molecule according to standard methods.

The nucleic acid sequences encoding acetylcholine transporter proteins or protein subsequences may be expressed in a variety of host cells, including E. coli, other bacterial hosts, yeast, and various higher eukaryotic cells such as the COS, CHO and HeLa cells lines and myeloma cell lines. In preferred embodiments, the acetylcholine transporter proteins are expressed in mammalian cells, e.g. rat pheochromocytoma PC12 cells. The recombinant protein gene will be operably linked to appropriate expression control sequences for each host. For E. coli this includes a promoter such as the T7, trp, or lambda promoters, a ribosome binding site and preferably a transcription termination signal. For eukaryotic cells, the control sequences will include a promoter and often an enhancer (e.g., an enhancer derived from immunoglobulin genes, SV40, cytomegalovirus, etc.), and a polyadenylation sequence, and may include splice donor and acceptor sequences.

The plasmids of the invention can be transferred into the chosen host cell by well-known methods such as calcium chloride transformation for E. coli and calcium phosphate treatment or electroporation for mammalian cells. In certain embodiments, cells are transfected in vivo using vectors commonly used in gene therapy applications.

One of skill would recognize that modifications can be made to the acetylcholine transporter proteins without diminishing their biological activity. Some modifications can be made to facilitate the cloning, expression, or incorporation of the targeting molecule into a fusion protein. Such modifications are well known to those of skill in the art and include, for example, a methionine added at the amino terminus to provide an initiation site, altered codon usage to facilitate expression, and the like.

As indicated above, nucleic acids encoding a heterologous acetylcholine transporter can be delivered in vivo to supplement cells in which such acetylcholine transport is deficient. Thus, in certain preferred embodiments, the nucleic acids encoding acetylcholine transporters are cloned into gene therapy vectors that are competent to transfect cells (such as human or other mammalian cells) in vitro and/or in vivo.

Many approaches for introducing nucleic acids into cells in vivo, ex vivo and in vitro are known. These include lipid or liposome based gene delivery (WO 96/18372; WO 93/24640; Mannino and Gould-Fogerite (1988) BioTechniques 6(7): 682-691; Rose U.S. Pat. No. 5,279,833; WO 91/06309; and Felgner et al. (1987) Proc. Natl. Acad. Sci. USA 84: 7413-7414) and replication-defective retroviral vectors harboring a therapeutic polynucleotide sequence as part of the retroviral genome (see, e.g., Miller et al. (1990) Mol. Cell. Biol. 10:4239 (1990); Kolberg (1992) J. NIH Res. 4: 43, and Cornetta et al. (1991) Hum. Gene Ther. 2: 215). “Gene therapy” procedures are discussed in greater detail below.

V. Altering Acetylcholine Transporter Expression/Activity.

In certain embodiments, this invention provides methods of inhibiting acetylcholine transport (e.g. uptake into synaptic vesicles) by a cell. Such methods preferably involve inhibiting expression or activity of an acetylcholine transporter (e.g. the C. elegans acetylcholine transporter and/or the homologues or orthologues thereof identified herein, etc.). In other embodimens, acetylcholine transporter expression or activity is upregulated (e.g. by transfecting cells with a construct that expresses a heterologous acetylcholine transporter, by altering the promoter, and the like).

acetylcholine transporter expression can upregulated or inhibited using a wide variety of approaches known to those of skill in the art. For example, methods of inhibiting acetylcholine transporter expression include, but are not limited to antisense molecules, acetylcholine transporter specific ribozymes, acetylcholine transporter specific catalytic DNAs, intrabodies directed against acetylcholine transporter proteins, RNAi, gene therapy approaches that knock out acetylcholine transporters, and small organic molecules that inhibit acetylcholine transporter expression/overexpression or block a receptor that is required to induce acetylcholine transporter expression. acetylcholine transporter expression and/or activity can be up-regulated by introducing constructs expressing acetylcholine transporter into the cell (e.g. using gene therapy approaches) or upregulating endogenous expression of acetylcholine transporter (e.g. using agents identified in the screening assays of this invention). It will be appreciated that the methods used to alter acetylcholine transporter expression/activity can generally also be used to alter expression/activity of acetylcholine transporter homologues.

A) Antisense Approaches.

Acetylcholine transporter gene expression can be downregulated or entirely inhibited by the use of antisense molecules. An “antisense sequence or antisense nucleic acid” is a nucleic acid that is complementary to the coding acetylcholine transporter mRNA nucleic acid sequence or a subsequence thereof. Binding of the antisense molecule to the acetylcholine transporter mRNA interferes with normal translation of the acetylcholine transporter polypeptide.

Thus, in accordance with preferred embodiments of this invention, preferred antisense molecules include oligonucleotides and oligonucleotide analogs that are hybridizable with acetylcholine transporter messenger RNA. This relationship is commonly denominated as “antisense.” The oligonucleotides and oligonucleotide analogs are able to inhibit the function of the RNA, either its translation into protein, its translocation into the cytoplasm, or any other activity necessary to its overall biological function. The failure of the messenger RNA to perform all or part of its function results in a reduction or complete inhibition of expression of acetylcholine transporter polypeptides.

In the context of this invention, the term “oligonucleotide” refers to a polynucleotide formed from naturally-occurring bases and/or cyclofuranosyl groups joined by native phosphodiester bonds. This term effectively refers to naturally-occurring species or synthetic species formed from naturally-occurring subunits or their close homologs. The term “oligonucleotide” may also refer to moieties which function similarly to oligonucleotides, but which have non naturally-occurring portions. Thus, oligonucleotides may have altered sugar moieties or inter-sugar linkages. Exemplary among these are the phosphorothioate and other sulfur containing species that are known for use in the art. In accordance with some preferred embodiments, at least one of the phosphodiester bonds of the oligonucleotide has been substituted with a structure which functions to enhance the ability of the compositions to penetrate into the region of cells where the RNA whose activity is to be modulated is located. It is preferred that such substitutions comprise phosphorothioate bonds, methyl phosphonate bonds, or short chain alkyl or cycloalkyl structures. In accordance with other preferred embodiments, the phosphodiester bonds are substituted with structures which are, at once, substantially non-ionic and non-chiral, or with structures which are chiral and enantiomerically specific. Persons of ordinary skill in the art will be able to select other linkages for use in the practice of the invention.

In one particularly preferred embodiment, the internucleotide phosphodiester linkage is replaced with a peptide linkage. Such peptide nucleic acids tend to show improved stability, penetrate the cell more easily, and show enhances affinity for their target. Methods of making peptide nucleic acids are known to those of skill in the art (see, e.g., U.S. Pat. Nos. 6,015,887, 6,015,710, 5,986,053, 5,977,296, 5,902,786, 5,864,010, 5,786,461, 5,773,571, 5,766,855, 5,736,336, 5,719,262, and 5,714,331).

Oligonucleotides may also include species that contain at least some modified base forms. Thus, purines and pyrimidines other than those normally found in nature may be so employed. Similarly, modifications on the furanosyl portions of the nucleotide subunits may also be effected, as long as the essential tenets of this invention are adhered to. Examples of such modifications are 2′-O-alkyl- and 2′-halogen-substituted nucleotides. Some specific examples of modifications at the 2′ position of sugar moieties which are useful in the present invention are OH, SH, SCH₃, F, OCH₃, OCN, O(CH₂)[n]NH₂ or O(CH₂)[n]CH₃, where n is from 1 to about 10, and other substituents having similar properties.

Such oligonucleotides are best described as being functionally interchangeable with natural oligonucleotides or synthesized oligonucleotides along natural lines, but which have one or more differences from natural structure. All such analogs are comprehended by this invention so long as they function effectively to hybridize with messenger RNA of acetylcholine transporter to inhibit the function of that RNA.

The oligonucleotides in accordance with this invention preferably comprise from about 3 to about 50 subunits. It is more preferred that such oligonucleotides and analogs comprise from about 8 to about 25 subunits and still more preferred to have from about 12 to about 20 subunits. As will be appreciated, a subunit is a base and sugar combination suitably bound to adjacent subunits through phosphodiester or other bonds. The oligonucleotides used in accordance with this invention may be conveniently and routinely made through the well-known technique of solid phase synthesis. Equipment for such synthesis is sold by several vendors, including Applied Biosystems. Any other means for such synthesis may also be employed, however, the actual synthesis of the oligonucleotides is well within the talents of the routineer. It is also will known to prepare other oligonucleotide such as phosphorothioates and alkylated derivatives.

Using the known sequence of the acetylcholine transporter gene(s)/cDNA(s) identified herein, appropriate and effective antisense oligonucleotide sequences can be readily determined.

B) Catalytic RNAs and DNAs

1) Ribozymes.

In another approach, acetylcholine transporter expression can be inhibited by the use of ribozymes. As used herein, “ribozymes” are include RNA molecules that contain anti-sense sequences for specific recognition, and an RNA-cleaving enzymatic activity. The catalytic strand cleaves a specific site in a target (acetylcholine transporter) RNA, preferably at greater than stoichiometric concentration. Two “types” of ribozymes are particularly useful in this invention, the hammerhead ribozyme (Rossi et al. (1991) Pharmac. Ther. 50: 245-254) and the hairpin ribozyme (Hampel et al. (1990) Nucl. Acids Res. 18: 299-304, and U.S. Pat. No. 5,254,678).

Because both hammerhead and hairpin ribozymes are catalytic molecules having antisense and endoribonucleotidase activity, ribozyme technology has emerged as a powerful extension of the antisense approach to gene inactivation. The ribozymes of the invention typically consist of RNA, but such ribozymes may also be composed of nucleic acid molecules comprising chimeric nucleic acid sequences (such as DNA/RNA sequences) and/or nucleic acid analogs (e.g., phosphorothioates).

Accordingly, within one aspect of the present invention ribozymes are provided which have the ability to inhibit acetylcholine transporter expression. Such ribozymes can be in the form of a “hammerhead” (for example, as described by Forster and Symons (1987) Cell 48: 211-220; Haseloff and Gerlach (1988) Nature 328: 596-600; Walbot and Bruening (1988) Nature 334: 196; Haseloff and Gerlach (1988) Nature 334: 585) or a “hairpin” (see, e.g. U.S. Pat. No. 5,254,678 and Hampel et al., European Patent Publication No. 0 360 257, published Mar. 26, 1990), and have the ability to specifically target, cleave acetylcholine transporter nucleic acids.

The sequence requirement for the hairpin ribozyme is any RNA sequence consisting of NNNBN*GUCNNNNNN (where N*G is the cleavage site, where B is any of G, C, or U, and where N is any of G, U, C, or A) (SEQ ID NO:3). Suitable sites fir recognition or target sequences for hairpin ribozymes can be readily determined from the acetylcholine transporter sequence(s) identified herein.

The preferred sequence at the cleavage site for the hammerhead ribozyme is any RNA sequence consisting of NUX (where N is any of G, U, C, or A and X represents C, U, or A) can be targeted. Accordingly, the same target within the hairpin leader sequence, GUC, is useful for the hammerhead ribozyme. The additional nucleotides of the hammerhead ribozyme or hairpin ribozyme is determined by the target flanking nucleotides and the hammerhead consensus sequence (see Ruffner et al. (1990) Biochemistry 29: 10695-10702).

Cech et al. (U.S. Pat. No. 4,987,071,) has disclosed the preparation and use of certain synthetic ribozymes which have endoribonuclease activity. These ribozymes are based on the properties of the Tetrahymena ribosomal RNA self-splicing reaction and require an eight base pair target site. A temperature optimum of 50° C. is reported for the endoribonuclease activity. The fragments that arise from cleavage contain 5′ phosphate and 3′ hydroxyl groups and a free guanosine nucleotide added to the 5′ end of the cleaved RNA. The preferred ribozymes of this invention hybridize efficiently to target sequences at physiological temperatures, making them particularly well suited for use in vivo.

The ribozymes of this invention, as well as DNA encoding such ribozymes and other suitable nucleic acid molecules can be chemically synthesized using methods well known in the art for the synthesis of nucleic acid molecules. Alternatively, Promega, Madison, Wis., USA, provides a series of protocols suitable for the production of RNA molecules such as ribozymes. The ribozymes also can be prepared from a DNA molecule or other nucleic acid molecule (which, upon transcription, yields an RNA molecule) operably linked to an RNA polymerase promoter, e.g., the promoter for T7 RNA polymerase or SP6 RNA polymerase. Such a construct may be referred to as a vector. Accordingly, also provided by this invention are nucleic acid molecules, e.g., DNA or cDNA, coding for the ribozymes of this invention. When the vector also contains an RNA polymerase promoter operably linked to the DNA molecule, the ribozyme can be produced in vitro upon incubation with the RNA polymerase and appropriate nucleotides. In a separate embodiment, the DNA may be inserted into an expression cassette (see, e.g., Cotten and Birnstiel (1989) EMBO J. 8(12):3861-3866; Hempel et al. (1989) Biochem. 28: 4929-4933, etc.).

After synthesis, the ribozyme can be modified by ligation to a DNA molecule having the ability to stabilize the ribozyme and make it resistant to RNase. Alternatively, the ribozyme can be modified to the phosphothio analog for use in liposome delivery systems. This modification also renders the ribozyme resistant to endonuclease activity.

The ribozyme molecule also can be in a host prokaryotic or eukaryotic cell in culture or in the cells of an organism/patient. Appropriate prokaryotic and eukaryotic cells can be transfected with an appropriate transfer vector containing the DNA molecule encoding a ribozyme of this invention. Alternatively, the ribozyme molecule, including nucleic acid molecules encoding the ribozyme, may be introduced into the host cell using traditional methods such as transformation using calcium phosphate precipitation (Dubensky et al. (1984) Proc. Natl. Acad. Sci., USA, 81: 7529-7533), direct microinjection of such nucleic acid molecules into intact target cells (Acsadi et al. (1991) Nature 352: 815-818), and electroporation whereby cells suspended in a conducting solution are subjected to an intense electric field in order to transiently polarize the membrane, allowing entry of the nucleic acid molecules. Other procedures include the use of nucleic acid molecules linked to an inactive adenovirus (Cotton et al. (1990) Proc. Natl. Acad. Sci., USA, 89:6094), lipofection (Felgner et al. (1989) Proc. Natl. Acad. Sci. USA 84: 7413-7417), microprojectile bombardment (Williams et al. (1991) Proc. Natl. Acad. Sci., USA, 88: 2726-2730), polycation compounds such as polylysine, receptor specific ligands, liposomes entrapping the nucleic acid molecules, spheroplast fusion whereby E. coli containing the nucleic acid molecules are stripped of their outer cell walls and fused to animal cells using polyethylene glycol, viral transduction, (Cline et al., (1985) Pharmac. Ther. 29: 69; and Friedmann et al. (1989) Science 244: 1275), and DNA ligand (Wu et al (1989) J. Biol. Chem. 264: 16985-16987), as well as psoralen inactivated viruses such as Sendai or Adenovirus. In one preferred embodiment, the ribozyme is introduced into the host cell utilizing a lipid, a liposome or a retroviral vector.

When the DNA molecule is operatively linked to a promoter for RNA transcription, the RNA can be produced in the host cell when the host cell is grown under suitable conditions favoring transcription of the DNA molecule. The vector can be, but is not limited to, a plasmid, a virus, a retrotransposon or a cosmid. Examples of such vectors are disclosed in U.S. Pat. No. 5,166,320. Other representative vectors include, but are not limited to adenoviral vectors (e.g., WO 94/26914, WO 93/9191; Kolls et al. (1994) PNAS 91(1):215-219; Kass-Eisler et al., (1993) Proc. Natl. Acad. Sci., USA, 90(24): 11498-502, Guzman et al. (1993) Circulation 88(6): 2838-48, 1993; Guzman et al. (1993) Cir. Res. 73(6): 1202-1207, 1993; Zabner et al. (1993) Cell 75(2): 207-216; Li et al. (1993) Hum Gene Ther. 4(4): 403-409; Caillaud et al. (1993) Eur. J. Neurosci. 5(10): 1287-1291), adeno-associated vector type 1 (“AAV-1”) or adeno-associated vector type 2 (“AAV-2”) (see WO 95/13365; Flotte et al. (1993) Proc. Natl. Acad. Sci., USA, 90(22):10613-10617), retroviral vectors (e.g., EP 0 415 731; WO 90/07936; WO 91/02805; WO 94/03622; WO 93/25698; WO 93/25234; U.S. Pat. No. 5,219,740; WO 93/11230; WO 93/10218) and herpes viral vectors (e.g., U.S. Pat. No. 5,288,641). Methods of utilizing such vectors in gene therapy are well known in the art, see, for example, Larrick and Burck (1991) Gene Therapy: Application of Molecular Biology, Elsevier Science Publishing Co., Inc., New York, N.Y., and Kreigler (1990) Gene Transfer and Expression: A Laboratory Manual, W.H. Freeman and Company, New York.

To produce ribozymes in vivo utilizing vectors, the nucleotide sequences coding for ribozymes are preferably placed under the control of a strong promoter such as the lac, SV40 late, SV40 early, or lambda promoters. Ribozymes are then produced directly from the transfer vector in vivo. Suitable transfector vectors for in vivo expression are discussed below.

2) Catalytic DNA

In a manner analogous to ribozymes, DNAs are also capable of demonstrating catalytic (e.g. nuclease) activity. While no such naturally-occurring DNAs are known, highly catalytic species have been developed by directed evolution and selection. Beginning with a population of 1014 DNAs containing 50 random nucleotides, successive rounds of selective amplification, enriched for individuals that best promote the Pb²⁺-dependent cleavage of a target ribonucleoside 3′-O—P bond embedded within an otherwise all-DNA sequence. By the fifth round, the population as a whole carried out this reaction at a rate of 0.2 min⁻¹. Based on the sequence of 20 individuals isolated from this population, a simplified version of the catalytic domain that operates in an intermolecular context with a turnover rate of 1 min⁻¹ (see, e.g., Breaker and Joyce (1994) Chem Biol 4: 223-229.

In later work, using a similar strategy, a DNA enzyme was made that could cleave almost any targeted RNA substrate under simulated physiological conditions. The enzyme is comprised of a catalytic domain of 15 deoxynucleotides, flanked by two substrate-recognition domains of seven to eight deoxynucleotides each. The RNA substrate is bound through Watson-Crick base pairing and is cleaved at a particular phosphodiester located between an unpaired purine and a paired pyrimidine residue. Despite its small size, the DNA enzyme has a catalytic efficiency (k_{cat}/Kₘ) of approximately 10⁹ M⁻¹min⁻¹ under multiple turnover conditions, exceeding that of any other known nucleic acid enzyme. By changing the sequence of the substrate-recognition domains, the DNA enzyme can be made to target different RNA substrates (Santoro and Joyce (1997) Proc. Natl. Acad. Sci., USA, 94(9): 4262-4266). Modifying the appropriate targeting sequences (e.g. as described by Santoro and Joyce, supra.) the DNA enzyme can easily be retargeted to ACETYLCHOLINE mRNA thereby acting like a ribozyme.

C) Knocking Out Acetylcholine Transporter(s).

In another approach, acetylcholine transporter can be nhibited/downregulated simply by “knocking out” the gene.

D) Acetylcholine Transporter Knockout Animals.

In certain embodiments, this invention provides animals in which acetylcholine transporters are “knocked out”. Such animals can be heterozygous or homozygous for the knockout.

Typically this is accomplished by disrupting the acetylcholine transporter gene(s), the promoter regulating the acetylcholine transporter gene(s) or sequences between the endogenous promoter(s) and the gene(s). Such disruption can be specifically directed to acetylcholine transporter nucleic acids by homologous recombination where a “knockout construct” contains flanking sequences complementary to the domain to which the construct is targeted. Insertion of the knockout construct (e.g. into an acetylcholine transporter gene) results in disruption of that gene.

The phrases “disruption of the gene” and “gene disruption” refer to insertion of a nucleic acid sequence into one region of the native DNA sequence (usually one or more exons) and/or the promoter region of a gene so as to decrease or prevent expression of that gene in the cell as compared to the wild-type or naturally occurring sequence of the gene. By way of example, a nucleic acid construct can be prepared containing a DNA sequence encoding an antibiotic resistance gene which is inserted into the DNA sequence that is complementary to the DNA sequence (promoter and/or coding region) to be disrupted. When this nucleic acid construct is then transfected into a cell, the construct will integrate into the genomic DNA. Thus, the cell and its progeny will no longer express the gene or will express it at a decreased level, as the DNA is now disrupted by the antibiotic resistance gene.

Knockout constructs can be produced by standard methods known to those of skill in the art. The knockout construct can be chemically synthesized or assembled, e.g., using recombinant DNA methods. The DNA sequence to be used in producing the knockout construct is digested with a particular restriction enzyme selected to cut at a location(s) such that a new DNA sequence encoding a marker gene can be inserted in the proper position within this DNA sequence. The proper position for marker gene insertion is that which will serve to prevent expression of the native acetylcholine transporter gene; this position will depend on various factors such as the restriction sites in the sequence to be cut, and whether an exon sequence or a promoter sequence, or both is (are) to be interrupted (i.e., the precise location of insertion necessary to inhibit promoter function or to inhibit synthesis of the native exon). Preferably, the enzyme selected for cutting the DNA will generate a longer arm and a shorter arm, where the shorter arm is at least about 300 base pairs (bp). In some cases, it will be desirable to actually remove a portion or even all of one or more exons of the gene to be suppressed so as to keep the length of the knockout construct comparable to the original genomic sequence when the marker gene is inserted in the knockout construct. In these cases, the genomic DNA is cut with appropriate restriction endonucleases such that a fragment of the proper size can be removed.

The marker gene can be any nucleic acid sequence that is detectable and/or assayable, however typically it is an antibiotic resistance gene or other gene whose expression or presence in the genome can easily be detected. The marker gene is usually operably linked to its own promoter or to another strong promoter from any source that will be active or can easily be activated in the cell into which it is inserted; however, the marker gene need not have its own promoter attached as it may be transcribed using the promoter of the gene to be suppressed. In addition, the marker gene will normally have a polyA sequence attached to the 3′ end of the gene; this sequence serves to terminate transcription of the gene. Preferred marker genes are any antibiotic resistance gene including, but not limited to neo (the neomycin resistance gene) and beta-gal (beta-galactosidase).

After the genomic DNA sequence has been digested with the appropriate restriction enzymes, the marker gene sequence is ligated into the genomic DNA sequence using methods well known to the skilled artisan (see, e.g., Berger and Kimmel, Guide to Molecular Cloning Techniques, Methods in Enzymology volume 152 Academic Press, Inc., San Diego, Calif.; Sambrook et al. (1989) Molecular Cloning—A Laboratory Manual (2nd ed.) Vol. 1-3, Cold Spring Harbor Laboratory, Cold Spring Harbor Press, NY; and Current Protocols in Molecular Biology, F. M. Ausubel et al., eds., Current Protocols, a joint venture between Greene Publishing Associates, Inc. and John Wiley & Sons, Inc., (1994) Supplement). The ends of the DNA fragments to be ligated are rendered compatible, e.g., by either cutting the fragments with enzymes that generate compatible ends, or by blunting the ends prior to ligation. Blunting is done using methods well known in the art, such as for example by the use of Klenow fragment (DNA polymerase I) to fill in sticky ends.

The production of knockout constructs and their use to produce knockout mice is well known to those of skill in the art (see, e.g., Dorfman et al. (1996) Oncogene 13: 925-931). The knockout constructs can be delivered to cells in vivo using gene therapy delivery vehicles (e.g. retroviruses, liposomes, lipids, dendrimers, etc.) as described above. Methods of knocking out genes are well described in the literature and essentially routine to those of skill in the art (see, e.g., Thomas et al. (1986) Cell 44(3): 419-428; Thomas, et al. (1987) Cell 51(3): 503-512)1; Jasin and Berg (1988) Genes & Development 2: 1353-1363; Mansour, et al. (1988) Nature 336: 348-352; Brinster, et al. (1989) Proc Natl Acad Sci 86: 7087-7091; Capecchi (1989) Trends in Genetics 5(3): 70-76; Frohman and Martin (1989) Cell 56: 145-147; Hasty, et al. (1991) Mol Cell Bio 11(11): 5586-5591; Jeannotte, et al. (1991) Mol Cell Biol. 11(11): 557814 5585; and Mortensen, et al. (1992) Mol Cell Biol. 12(5): 2391-2395.

The use of homologous recombination to alter expression of endogenous genes is also described in detail in U.S. Pat. No. 5,272,071, WO 91/09955, WO 93/09222, WO 96/29411, WO 95/31560, and WO 91/12650.

Production of the knockout animals of this invention is not dependent on the availability of ES cells. In various embodiments, knockout animals of this invention can be produced using methods of somatic cell nuclear transfer. In preferred embodiments using such an approach, a somatic cell is obtained from the species in which the acetylcholine transporter gene is to be knocked out. The cell is transfected with a construct that introduces a disruption in the acetylcholine transporter gene (e.g. via heterologous recombination) as described herein. Cells harboring a knocked out acetylcholine transporter gene are selected as described herein. The nucleus of such cells harboring the knockout is then placed in an unfertilized enucleated egg (e.g., eggs from which the natural nuclei have been removed by microsurgery). Once the transfer is complete, the recipient eggs contained a complete set of genes, just as they would if they had been fertilized by sperm. The eggs are then cultured for a period before being implanted into a host mammal (of the same species that provided the egg) where they are carried to term, culminating in the berth of a transgenic animal comprising a nucleic acid construct containing one or more disrupted acetylcholine transporter genes.

The production of viable cloned mammals following nuclear transfer of cultured somatic cells has been reported for a wide variety of species including, but not limited to frogs (McKinnell (1962) J. Hered. 53, 199-207), calves (Kato et al. (1998) Science 262: 2095-2098), sheep (Campbell et al. (1996) Nature 380: 64-66), mice (Wakayamaand Yanagimachi (1999) Nat. Genet. 22: 127-128), goats (Baguisi et al. (1999) Nat. Biotechnol. 17: 456-461), monkeys (Meng et al. (1997) Biol. Reprod. 57: 454-459), and pigs (Bishop et al. (2000) Nature Biotechnology 18: 1055-1059). Nuclear transfer methods have also been used to produce clones of transgenic animals. Thus, for example, the production of transgenic goats carrying the human antithrobin III gene by somatic cell nuclear transfer has been reported (Baguisi et al. (1999) Nature Biotechnology 17: 456-461).

Using methods of nuclear transfer as described in these and other references, cell nuclei derived from differentiated fetal or adult, mammalian cells are transplanted into enucleated mammalian oocytes of the same species as the donor nuclei. The nuclei are reprogrammed to direct the development of cloned embryos, which can then be transferred into recipient females to produce fetuses and offspring, or used to produce cultured inner cell mass (CICM) cells. The cloned embryos can also be combined with fertilized embryos to produce chimeric embryos, fetuses and/or offspring.

Somatic cell nuclear transfer also allows simplification of transgenic procedures by working with a differentiated cell source that can be clonally propagated. This eliminates the need to maintain the cells in an undifferentiated state, thus, genetic modifications, both random integration and gene targeting, are more easily accomplished. Also by combining nuclear transfer with the ability to modify and select for these cells in vitro, this procedure is more efficient than previous transgenic embryo techniques.

Nuclear transfer techniques or nuclear transplantation techniques are known in the literature. See, in particular, Campbell et al. (1995) Theriogenology, 43:181; Collas et al. (1994) Mol. Report Dev., 38:264-267; Keefer et al. (1994) Biol. Reprod., 50:935-939; Sims et al. (1993) Proc. Natl. Acad. Sci., USA, 90:6143-6147; WO 94/26884; WO 94/24274, WO 90/03432, U.S. Pat. Nos. 5,945,577, 4,944,384, 5,057,420 and the like.

E) Intrabodies.

In still another embodiment, acetylcholine transporter expression/activity is inhibited by transfecting the subject cell(s) (e.g., cells of the vascular endothelium) with a nucleic acid construct that expresses an intrabody. An intrabody is an intracellular antibody, in this case, capable of recognizing and binding to an acetylcholine transporter polypeptide. The intrabody is expressed by an “antibody cassette”, containing a sufficient number of nucleotides coding for the portion of an antibody capable of binding to the target (acetylcholine transporter polypeptide) operably linked to a promoter that will permit expression of the antibody in the cell(s) of interest. The construct encoding the intrabody is delivered to the cell where the antibody is expressed intracellularly and binds to the target acetylcholine transporter, thereby disrupting the target from its normal action. This antibody is sometimes referred to as an “intrabody”.

In one preferred embodiment, the “intrabody gene” (antibody) of the antibody cassette would utilize a cDNA, encoding heavy chain variable (V_{H}) and light chain variable (V_{L}) domains of an antibody which can be connected at the DNA level by an appropriate oligonucleotide as a bridge of the two variable domains, which on translation, form a single peptide (referred to as a single chain variable fragment, “sFv”) capable of binding to a target such as an acetylcholine transporter protein. The intrabody gene preferably does not encode an operable secretory sequence and thus the expressed antibody remains within the cell.

Anti-acetylcholine transporter antibodies suitable for use/expression as intrabodies in the methods of this invention can be readily produced by a variety of methods. Such methods include, but are not limited to, traditional methods of raising “whole” polyclonal antibodies, which can be modified to form single chain antibodies, or screening of, e.g. phage display libraries to select for antibodies showing high specificity and/or avidity for acetylcholine transporter. Such screening methods are described above in some detail.

The antibody cassette is delivered to the cell by any of the known means. This discloses the use of a fusion protein comprising a target moiety and a binding moiety. The target moiety brings the vector to the cell, while the binding moiety carries the antibody cassette. Other methods include, for example, Miller (1992) Nature 357: 455-460; Anderson (1992) Science 256: 808-813; Wu, et al. (1988) J. Biol. Chem. 263: 14621-14624. For example, a cassette containing these (anti-acetylcholine transporter) antibody genes, such as the sFv gene, can be targeted to a particular cell by a number of techniques including, but not limited to the use of tissue-specific promoters, the use of tissue specific vectors, and the like. Methods of making and using intrabodies are described in detail in U.S. Pat. No. 6,004,940.

E) Small Organic Molecules.

In still another embodiment, acetylcholine transporter expression and/or acetylcholine transporter protein activity can be inhibited (or upregulated) by the use of small organic molecules. Such molecules include, but are not limited to molecules that specifically bind to the DNA comprising the acetylcholine transporter promoter and/or coding region, molecules that bind to and complex with acetylcholine transporter mRNA, molecules that inhibit the signaling pathway that results in acetylcholine transporter upregulation, and molecules that bind to and/or compete with acetylcholine transporter polypeptides. Small organic molecules effective at inhibiting acetylcholine transporter expression can be identified with routine screening using the methods described herein.

The methods of inhibiting acetylcholine transporter expression described above are meant to be illustrative and not limiting. In view of the teachings provided herein, other methods of inhibiting acetylcholine transporter will be known to those of skill in the art.

F) Modes of Administration.

The mode of administration of the acetylcholine transporter blocking (or upregulating) agent depends on the nature of the particular agent. Antisense molecules, catalytic RNAs (ribozymes), catalytic DNAs, small organic molecules, and other molecules (e.g. lipids, antibodies, etc.) used as acetylcholine transporter inhibitors may be formulated as pharmaceuticals (e.g. with suitable excipient) and delivered using standard pharmaceutical formulation and delivery methods as described below. Antisense molecules, catalytic RNAs (ribozymes), catalytic DNAs, and additionally, knockout constructs, and constructs encoding intrabodies can be delivered and (if necessary) expressed in target cells (e.g. vascular endothelial cells) using methods of gene therapy, e.g. as described below.

1) Pharmaceutical Administration.

In order to carry out the methods of the invention, one or more modulators (e.g. inhibitors or agonists) of acetylcholine transporter expression (e.g. ribozymes, antibodies, antisense molecules, small organic molecules, etc.) are administered to an individual to ameliorate one or more symptoms of a neurological dysfunction (e.g. Alzheimers, ALS, stroke, epilepsy, etc.). While this invention is described generally with reference to human subjects, veterinary applications are contemplated within the scope of this invention.

Various inhibitors or upregulators may be administered, if desired, in the form of salts, esters, amides, prodrugs, derivatives, and the like, provided the salt, ester, amide, prodrug or derivative is suitable pharmacologically, i.e., effective in the present method. Salts, esters, amides, prodrugs and other derivatives of the active agents may be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

The acetylcholine transporter inhibitors or upregulators and various derivatives and/or formulations thereof are useful for parenteral, topical, oral, or local administration, such as by aerosol or transdermally, for prophylactic and/or therapeutic treatment of coronary disease and/or rheumatoid arthritis. The pharmaceutical compositions can be administered in a variety of unit dosage forms depending upon the method of administration. Suitable unit dosage forms, include, but are not limited to powders, tablets, pills, capsules, lozenges, suppositories, etc.

The acetylcholine transporter inhibitors or upregulators and various derivatives and/or formulations thereof are typically combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers.

Other physiologically acceptable compounds include wetting agents, emulsifying agents, dispersing agents or preservatives which are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s). The excipients are preferably sterile and generally free of undesirable matter. These compositions may be sterilized by conventional, well known sterilization techniques.

The concentration of active agent(s) in the formulation can vary widely, and will be selected primarily based on fluid volumes, viscosities, body weight and the like in accordance with the particular mode of administration selected and the patient's needs.

In therapeutic applications, the compositions of this invention are administered to a patient suffering from a disease (e.g., atherosclerosis and/or associated conditions, and/or rheumatoid arthritis) in an amount sufficient to cure or at least partially arrest the disease and/or its symptoms (e.g. to reduce plaque formation, to reduce monocyte recruitment, etc.) An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the active agents of the formulations of this invention to effectively treat (ameliorate one or more symptoms) the patient.

In certain preferred embodiments, the acetylcholine transporter inhibitors or upregulators are administered orally (e.g. via a tablet) or as an injectable in accordance with standard methods well known to those of skill in the art. In other preferred embodiments, the acetylcholine transporter inhibitors or upregulators can also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

The foregoing formulations and administration methods are intended to be illustrative and not limiting. It will be appreciated that, using the teaching provided herein, other suitable formulations and modes of administration can be readily devised.

2) Gene Therapy.

As indicated above, molecules encoding and expressing heterologous acetylcholine transporter, antisense molecules, catalytic RNAs (ribozymes), catalytic DNAs, and additionally, knockout constructs, and constructs encoding intrabodies can be delivered and transcribed and/or expressed in target cells (e.g. cancer cells) using methods of gene therapy. Thus, in certain preferred embodiments, the nucleic acids encoding knockout constructs, intrabodies, antisense molecules, catalytic RNAs or DNAs, etc. are cloned into gene therapy vectors that are competent to transfect cells (such as human or other mammalian cells) in vitro and/or in vivo.

Many approaches for introducing nucleic acids into cells in vivo, ex vivo and in vitro are known. These include lipid or liposome based gene delivery (WO 96/18372; WO 93/24640; Mannino and Gould-Fogerite (1988) BioTechniques 6(7): 682-691; Rose U.S. Pat. No. 5,279,833; WO 91/06309; and Felgner et al. (1987) Proc. Natl. Acad. Sci. USA 84: 7413-7414) and replication-defective retroviral vectors harboring a therapeutic polynucleotide sequence as part of the retroviral genome (see, e.g., Miller et al. (1990) Mol. Cell. Biol. 10:4239 (1990); Kolberg (1992) J. NIH Res. 4: 43, and Cornetta et al. (1991) Hum. Gene Ther. 2: 215).

For a review of gene therapy procedures, see, e.g., Anderson, Science (1992) 256: 808-813; Nabel and Felgner (1993) TIBTECH 11: 211-217; Mitani and Caskey (1993) TIBTECH 11: 162-166; Mulligan (1993) Science, 926-932; Dillon (1993) TIBTECH 11: 167-175; Miller (1992) Nature 357: 455-460; Van Brunt (1988) Biotechnology 6(10): 1149-1154; Vigne (1995) Restorative Neurology and Neuroscience 8: 35-36; Kremer and Perricaudet (1995) British Medical Bulletin 51(1) 31-44; Haddada et al. (1995) in Current Topics in Microbiology and Immunology, Doerfler and Bohm (eds) Springer-Verlag, Heidelberg Germany; and Yu et al., (1994) Gene Therapy, 1:13-26.

Widely used retroviral vectors include those based upon murine leukemia virus (MuLV), gibbon ape leukemia virus (GaLV), Simian Immunodeficiency virus (SIV), human immunodeficiency virus (HIV), alphavirus, and combinations thereof (see, e.g., Buchscher et al. (1992) J. Virol. 66(5) 2731-2739; Johann et al. (1992) J. Virol. 66 (5):1635-1640 (1992); Sommerfelt et al., (1990) Virol. 176:58-59; Wilson et al. (1989) J. Virol. 63:2374-2378; Miller et al., J. Virol. 65:2220-2224 (1991); Wong-Staal et al., PCT/US94/05700, and Rosenburg and Fauci (1993) in Fundamental Immunology, Third Edition Paul (ed) Raven Press, Ltd., New York and the references therein, and Yu et al. (1994) Gene Therapy, supra; U.S. Pat. No. 6,008,535, and the like).

The vectors are optionally pseudotyped to extend the host range of the vector to cells which are not infected by the retrovirus corresponding to the vector. For example, the vesicular stomatitis virus envelope glycoprotein (VSV-G) has been used to construct VSV-G-pseudotyped HIV vectors which can infect hematopoietic stem cells (Naldini et al. (1996) Science 272:263, and Akkina et al. (1996) J Virol 70:2581).

Adeno-associated virus (AAV)-based vectors are also used to transduce cells with target nucleic acids, e.g., in the in vitro production of nucleic acids and peptides, and in in vivo and ex vivo gene therapy procedures. See, West et al. (1987) Virology 160:38-47; Carter et al. (1989) U.S. Pat. No. 4,797,368; Carter et al. WO 93/24641 (1993); Kotin (1994) Human Gene Therapy 5:793-801; Muzyczka (1994) J. Clin. Invst. 94:1351 for an overview of AAV vectors. Construction of recombinant AAV vectors are described in a number of publications, including Lebkowski, U.S. Pat. No. 5,173,414; Tratschin et al. (1985) Mol. Cell. Biol. 5(11):3251-3260; Tratschin, et al. (1984) Mol. Cell. Biol., 4: 2072-2081; Hermonat and Muzyczka (1984) Proc. Natl. Acad. Sci. USA, 81: 6466-6470; McLaughlin et al. (1988) and Samulski et al. (1989) J. Virol., 63:03822-3828. Cell lines that can be transformed by rAAV include those described in Lebkowski et al. (1988) Mol. Cell. Biol., 8:3988-3996. Other suitable viral vectors include, but are not limited to, herpes virus, lentivirus, and vaccinia virus.

V. Kits.

In still another embodiment, this invention provides kits for the practice of the methods of this invention. In certain embodiments the kits comprise a nucleic acid that encodes an acetylcholine transporter transporter (e.g. the C. elegans acetylcholine transporter and/or the homologues or orthologues thereof identified herein) and/or an antibody that specifically binds to an acetylcholine transporter, and/or a cell expressing an endogenous acetylcholine transporter, and/or a cell transfected with a heterologous nucleic acid capable of expressing a acetylcholine transporter. In certain embodiments, the kit comprises a cell and a vector suitable for transfecting the cell with a heterologous nucleic acid capable of expressing an acetylcholine transporter. In certain embodiments, the kit comprises a nucleic acid probe that can specifically hybridize to a nucleic acid encoding an acetylcholine transporter. The probe can, optionally, be labeled with a detectable label, e.g., as described herein. In certain embodiments, the kit comprises a vector comprising an expression cassette that expresses an acetylcholine transporter. In certain preferred embodiments, the vector is one that permits in vivo transfection of a cell. The kit can optionally include various transfection reagents, (e.g. cationic lipids, dendrimers, and the like).

The kits can optionally include any reagents and/or apparatus to facilitate practice of the methods described herein. Such reagents include, but are not limited to buffers, instrumentation (e.g. bandpass filter), reagents for detecting a signal from a detectable label, transfection reagents, cell lines, vectors, and the like.

In addition, the kits can include instructional materials containing directions (i.e., protocols) for the practice of the methods of this invention. Preferred instructional materials provide protocols for utilizing the kit contents for screening for agents that increase or decrease acetylcholine transporter expression and/or activity, e.g. as described herein. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media can include addresses to internet sites that provide such instructional materials.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A method of screening for an agent that modulates activity of a cholinergic synapse, said method comprising:
i) contacting a cell comprising a nucleic acid encoding an acetylcholine transporter with a test agent; and
ii) detecting expression or activity of said acetylcholine transporter, where an increase or decrease in the expression or activity of the acetylcholine transporter as compared to a control indicates that said test agent modulates the activity of a cholinergic synapse.

2. The method of claim 1, wherein said control is a negative control comprising contacting a cell at a lower concentration of said test agent.

3. The method of claim 2, wherein said lower concentration is the absence of said test agent.

4. The method of claim 1, wherein said cell is a somatic cell.

5. The method of claim 1, wherein said cell is an oocyte.

6. The method of claim 1, wherein said cell is a nerve cell.

7. The method of claim 1, wherein said cell is a vertebrate cell.

8. The method of claim 7, wherein said cell is a mammalian cell.

9. The method of claim 7, wherein said cell is a human cell.

10. The method of claim 1, wherein said detecting comprises detecting an acetylcholine transporter nucleic acid.

11. The method of claim 1, wherein said detecting comprises detecting a an acetylcholine transporter polypeptide.

12. The method of claim 1, wherein said detecting comprises measuring activity of an acetylcholine transporter polypeptide.

13. The method of claim 10, wherein said detecting acetylcholine transporter nucleic acid. comprises performing a nucleic acid hybridization.

14. The method of claim 10, wherein said detecting a acetylcholine transporter nucleic acid. comprises a method selected from the group consisting of a Northern blot, a Southern blot using DNA derived from the acetylcholine transporter mRNA, an array hybridization, an affinity chromatography, and an in situ hybridization.

15. The method of claim 10, wherein said detecting a acetylcholine transporter nucleic acid. comprises a nucleic acid amplification.

16. The method of claim 11, wherein said detecting an acetylcholine transporter polypeptide comprises a method selected from the group consisting of capillary electrophoresis, Western blot, mass spectroscopy, ELISA, immunochromatography, thin layer chromatography, and immunohistochemistry.

17. The method of claim 12, wherein said measuring activity of a acetylcholine transporter polypeptide activity comprises detecting acetylcholine transport in a cell expressing a heterologous acetylcholine transporter polypeptide.

18. The method of claim 1, wherein said test agent is not an antibody.

19. The method of claim 1, wherein said test agent is not a nucleic acid.

20. The method of claim 1, wherein said test agent is not a protein.

21. The method of claim 1, wherein said test agent is a small organic molecule.

22. The method of claim 1, wherein said acetylcholine transporter is a C. elegans acetylcholine transporter.

23. The method of claim 1, wherein said acetylcholine transporter is an orthologue of a C. elegans acetylcholine transporter.

24. The method of claim 1, wherein said acetylcholine transporter is a human acetylcholine transporter.

25. A method of prescreening for a potential modulator of cholinergic synaptic activity, said method comprising:
contacting an acetylcholine transporter polypeptide or a nucleic acid encoding an acetylcholine transporter polypeptide with a test agent; and
detecting binding of said test agent to said acetylcholine transporter polypeptide or to said nucleic acid encoding an acetylcholine transporter polypeptide wherein specific binding of said test agent to the acetylcholine transporter polypeptide or acetylcholine transporter nucleic acid indicates that said test agent is a potential modulator of cholinergic synaptic.

26. The method of claim 25, further comprising recording test agents that specifically bind to said acetylcholine transporter polypeptide or to said nucleic acid encoding an acetylcholine transporter polypeptide in a database of candidate modulators of cholinergic synaptic activity.

27. The method of claim 25, wherein said acetylcholine transporter is a C. elegans acetylcholine transporter.

28. The method of claim 25, wherein said acetylcholine transporter is an orthologue of a C. elegans acetylcholine transporter.

29. The method of claim 25, wherein said acetylcholine transporter is a human acetylcholine transporter.

30. The method of claim 25, wherein said test agent is not an antibody.

31. The method of claim 25, wherein said test agent is not a protein.

32. The method of claim 25, wherein said detecting comprises detecting specific binding of said test agent to said nucleic acid encoding an acetylcholine transporter polypeptide.

33. The method of claim 32, wherein said binding is detected using a method selected from the group consisting of a Northern blot, a Southern blot using DNA derived from an acetylcholine transporter mRNA, an array hybridization, an affinity chromatography, and an in situ hybridization.

34. The method of claim 25, wherein said detecting comprises detecting specific binding of said test agent to said acetylcholine transporter polypeptide.

35. The method of claim 48, wherein said detecting is via a method selected from the group consisting of capillary electrophoresis, a Western blot, mass spectroscopy, ELISA, immunochromatography, thin layer chromatography, and immunohistochemistry.

36. The method of claim 25, wherein said test agent is contacted directly to said acetylcholine transporter polypeptide or to said nucleic acid encoding an acetylcholine transporter polypeptide.

37. The method of claim 25, wherein said test agent is contacted to a cell containing said acetylcholine transporter polypeptide or to said nucleic acid encoding an acetylcholine transporter polypeptide.

38. The method of claim 37, wherein said cell is cultured ex vivo.

39. A cell comprising a heterologous nucleic acid encoding an acetylcholine transporter.

40. The cell of claim 39, wherein said cell is a mammalian cell.

41. The cell of claim 39, wherein said cell is a somatic cell.

42. The cell of claim 39, wherein said cell is an oocyte or a nerve cell.

43. The cell of claim 39, wherein said cell transports acetylcholine via said acetylcholine transporter.

44. The method of claim 37, wherein said acetylcholine transporter is a C. elegans acetylcholine transporter.

45. The method of claim 37, wherein said acetylcholine transporter is an orthologue of a C. elegans acetylcholine transporter.

46. The method of claim 37, wherein said acetylcholine transporter is a human acetylcholine transporter.

47. A method of increasing acetylcholine transport by a mammalian cell, said method comprising transfecting said cell with a nucleic acid encoding an acetylcholine transporter.

48. The method of claim 47, wherein said nucleic acid encoding nucleic acid encoding an acetylcholine transporter is operably linked to a constitutive promoter.

49. The method of claim 47, wherein said nucleic acid encoding an acetylcholine transporter is operably linked to an inducible promoter.

50. The method of claim 47, wherein said nucleic acid encoding an acetylcholine transporter is operably linked to a tissue-specific promoter.

51. A kit for screening for compounds that modulate acetylcholine transport, said kit comprising
a cell that expresses an acetylcholine transporter; and
a detection moiety selected from the group consisting of an antibody that specifically binds to acetylcholine transporter, a nucleic acid that specifically binds to a nucleic acid encoding said acetylcholine transporter, a primer that specifically amplifies a nucleic acid encoding said acetylcholine transporter or a fragment thereof, and a labeled acetylcholine.

52. The kit of claim 51, wherein said cell is a cell comprising a heterologous nucleic acid encoding said acetylcholine transporter.

53. The kit of claim 51, further comprising instructional materials providing protocols for screening for modulators of an acetylcholine transporter and teaching that such modulators alters acetylcholine transport.

54. An isolated nucleic acid encoding an acetylcholine transporter.

55. The isolated nucleic acid of claim 54, wherein said nucleic acid encodes a C. elegans acetylcholine transporter.

56. The isolated nucleic acid of claim 54, wherein said nucleic acid encodes an orthologue of a C. elegans acetylcholine transporter.

57. The isolated nucleic acid of claim 54, wherein said nucleic acid encodes a human acetylcholine transporter.

58. An isolated protein comprising an acetylcholine transporter.

59. The isolated protein of claim 58, wherein said transporter is a C. elegans acetylcholine transporter.

60. The isolated protein of claim 58, wherein said transporter is an orthologue of a C. elegans acetylcholine transporter.

61. The isolated protein of claim 58, wherein said transporter is a human acetylcholine transporter.

62. A cell expressing a heterologous protein wherein said heterologous protein is an acetylcholine transporter.

63. The cell of claim 62, wherein said acetylcholine transporter is a C. elegans acetylcholine transporter.

64. The cell of claim 62, wherein said acetylcholine transporter is an orthologue of a C. elegans acetylcholine transporter.

65. The cell of claim 62, wherein said acetylcholine transporter is a human acetylcholine transporter.

66. An antibody that specifically binds an acetylcholine transporter.

67. The antibody of claim 66, wherein said antibody specifically binds a C. elegans acetylcholine transporter.

68. The antibody of claim 66, wherein said antibody specifically binds a human acetylcholine transporter.

69. The antibody of claim 66, wherein said antibody is a monoclonal antibody.

70. The antibody of claim 66, wherein said antibody is a single chain antibody.

71. A method of modulating the activity of a cholinergic synapse, said method comprising altering the expression or activity of an acetylcholine transporter.

72. The method of claim 71, wherein said acetylcholine transporter is a C. elegans acetylcholine transporter.

73. The method of claim 71, wherein said acetylcholine transporter is an orthologue of a C. elegans acetylcholine transporter.

74. The method of claim 71, wherein said acetylcholine transporter is a human acetylcholine transporter.


SEQUENCE LISTING (3 sequences)

SEQ ID NO: 1 - DNA, 2145 bp, Caenorhabditis elegans
atgtcggtttcgagcaacgatccggagcagaggaatggacggggcatggcaagcggaaacaacgtggatatgagcctatatcctccgtttattaagcagctcgacgcaaaattgcccgattacacacgagaaggtgacatcgaatatccattcgaagagattacgggtgtcggagacgaaaataggatacgaggaaattggagtaacaagtcggattatctgctagcagtaattggtttcacagcgggagttggaagtttctggaaattcccatttctagtattccaacatggcggtgcagcattccttgtaccatatttatgtatgttatgcctcgcctcactaccaatgttctttatggaaatggtacttggacagttctcctcgtcggctgcgattagtgtttggaaggtggttccgttgtttaagggaatcggcttcgctcaagtgacaatttcgggattttttgcggttttcttcaatataatctctgcgtggacactcttctacctgataaattcgttcagcttctcgattccatggtcaaattgtgcaaattcgtggtccggagaaaactgtacacttggcacccgaatccagtgtaaagagatgaatgggacacttttagtgaatggaagttgtattgtggagcacgcgagctccaacgaaactacagtaatcccacttcatgatcttggctcaataccgagcttgaagtattttcacaacgacgtattgatgttatcaaagggagtggacgattttggaacattaaattggtatctcggattatgtgtacttgcatgctggattgcagtctttttatgcctatttcaaggtgttaaatccagtggaaaagtagtctacgtggccgtgatagtaccattcatcatacttacagtactcctcacccgcctactcactttagacggaagtctcgccgcagttttctattttttaacgccgaaatgggaaatactaatggatttgcacgtttggggtgaagcggctgttcaggcgttctattcggtttcgtgttgttctggtggactatttacaattgccagttattcacgatttcataataatatttataaagacatctggctcgtgctcatcgttgacgtgatcgtttcgctagtcggttgcctacttaccttctctgcaatcggattcacctgctacgaattcgccatctcgcttgacaaattccatatccgagatggtttccatttggtcttcgtattcctagcagaagcgttagccggtgtatcagttgctccactttacgcaggactattcttcattatgattctgctcgttgtccatgcaacccaaatgtttgtcgtcgaaacgatcgtctcatcgatttgtgatgaatatccggaacgtcttcgtcgaaatcgtcgccacgtattaaccactgtctgtgctctcttcattctgctctctattccattttgtctctcatctgggctcttctggatggagcttctcacccaatttgtgctcacttggccactggttgtcattgcatttttagagtgtatggctattaattgggtatacggagttgataatatgctggataatgctaaatggattgttggctactggccgccgtgttatattttttggaagattttattcaaattcatttgtcctatggtatatctggcgattctttgcttcctttggcttgattggaactcaattcaatacgaatcctatcaattcccgtattggtcaattctgacagcatggtgcatcgccagttttccgctaatcctcattccaatcgtcggaatctggcaattttgtatcgctaagggtacaattacccaaaaatggtggagggtactgtacccggacgacgcttggggacccgcgatggcaatacatcgagccgaaaaatttccgctacaaattccagaggcccggaggttgctgctgccgccagaagtcgaaattgccagttcgcggggggttttacaggaagaaatgccaatgtcctacgactacaacacatcctccgctgctgacgtccgctcaaatcgatcaactggacatggtgcaaccgacgtgcggagcgtcgccgccacgaacaacacaattccgaaatttgagcgagaaactgcgatctag

SEQ ID NO: 2 - protein, 714 aa, Caenorhabditis elegans
MSVSSNDPEQRNGRGMASGNNVDMSLYPPFIKQLDAKLPDYTREGDIEYPFEEITGVGDENRIRGNWSNKSDYLLAVIGFTAGVGSFWKFPFLVFQHGGAAFLVPYLCMLCLASLPMFFMEMVLGQFSSSAAISVWKVVPLFKGIGFAQVTISGFFAVFFNIISAWTLFYLINSFSFSIPWSNCANSWSGENCTLGTRIQCKEMNGTLLVNGSCIVEHASSNETTVIPLHDLGSIPSLKYFHNDVLMLSKGVDDFGTLNWYLGLCVLACWIAVFLCLFQGVKSSGKVVYVAVIVPFIILTVLLTRLLTLDGSLAAVFYFLTPKWEILMDLHVWGEAAVQAFYSVSCCSGGLFTIASYSRFHNNIYKDIWLVLIVDVIVSLVGCLLTFSAIGFTCYEFAISLDKFHIRDGFHLVFVFLAEALAGVSVAPLYAGLFFIMILLVVHATQMFVVETIVSSICDEYPERLRRNRRHVLTTVCALFILLSIPFCLSSGLFWMELLTQFVLTWPLVVIAFLECMAINWVYGVDNMLDNAKWIVGYWPPCYIFWKILFKFICPMVYLAILCFLWLDWNSIQYESYQFPYWSILTAWCIASFPLILIPIVGIWQFCIAKGTITQKWWRVLYPDDAWGPAMAIHRAEKFPLQIPEARRLLLPPEVEIASSRGVLQEEMPMSYDYNTSSAADVRSNRSTGHGATDVRSVAATNNTIPKFERETAI

SEQ ID NO: 3 - RNA, 14 nt, Artificial
  Synthetic hairpin ribozyme
nnnbngucnnnnnn